(19) United States
(12) Patent Application Publication
(10) Pub. No.: US 2023/0192774 A1
(43) Pub. Date: Jun. 22, 2023

(54) IMMUNOTHERAPY FOR POLYOMAVIRUSES

(71) Applicant: The Council of the Queensland Institute of Medical Research, Herston (AU)

(72) Inventors: Rajiv KHANNA, Herston (AU); George Robin Ambalathingal THOMAS, St. Lucia (AU)

(21) Appl. No.: 17/581,617

(22) Filed: Jan. 21, 2022

Related U.S. Application Data
(63) Continuation of application No. PCT/IB2020/000606, filed on Jul. 23, 2020.
(60) Provisional application No. 62/878,105, filed on Jul. 24, 2019.

(51) Int. Cl. C07K 14/005; G01N 33/50; C12Q 1/70; C12N 5/0783

(52) U.S. Cl. CPC C07K 14/005; G01N 33/505; C12Q 1/70; C12N 5/0638; C12N 2710/22022; C12N 2710/22034; C12N 2501/2302; C12N 2501/2307; C12N 2501/2312; C12N 2501/2315; G01N 2333/01; G01N 2800/26; A61K 39/00

(57) ABSTRACT

Provided herein are methods and compositions related to polyomavirus epitopes useful in the treatment of cancer or a polyomavirus infection.

### RELATED APPLICATIONS

This application is a continuation of International Application No. PCT/IB2020/000606 filed Jul. 23, 2020, which claims the benefit of priority to U.S. Provisional Patent Application Ser. No. 62/878,105 filed Jul. 24, 2019, each of which is incorporated by reference in its entirety.

### INCORPORATION BY REFERENCE OF SEQUENCE LISTING

The instant application contains a Sequence Listing which has been submitted electronically in ASCII format and is hereby incorporated by reference in its entirety. Said ASCII copy, created on Dec. 19, 2022, is named 3684727_502USPC_00565248_SL.txt and is 31,542 bytes in size.

### BACKGROUND

Polyomaviruses are ubiquitous viruses that infect a wide range of mammalian species. Currently, more than 12 distinct human polyomavirus species have been identified, including BK polyomavirus (BKV/Human polyomavirus 1), John Cunningham polyomavirus (JCV/Human polyomavirus 2), and Merkel cell polyomavirus (MCV/Human polyomavirus 5).

Most such polyomaviruses are typically asymptomatic in humans. However, those human polyomaviruses associated with diseases are often acquired in childhood and/or in immunocompromised hosts. For example, initial JCV infection may occur via the tonsils or the gastrointestinal tract and remain latent in the gastrointestinal tract, and possibly in the lymphoid organs, neuronal tissue, and kidney, where virus continues to reproduce and shed viral particles. Subsequently, under the circumstances of immuno-incompetence, immunosuppression, or immunodeficiency, both JCV and BKV may reactivate and progress to significant organ disease.

Of particular note is JC virus, which can cross the blood—brain barrier into the central nervous system (CNS) where it is neurotropic, infecting glial cells (e.g., oligodendrocytes and astrocytes) and meningeal cells. Once reactivated in the brain (e.g., in an immunocompromised subject), JCV infection is associated with white matter demyelination and several pathological syndromes, such as JCV granule cell layer neuronopathy (JCV GCN), JCV encephalopathy (JCV CPN/JCVE), JCV meningitis (JCVM), and especially progressive multifocal leukoencephalopathy (PML), a demyelinating disease of the central nervous system with a high mortality rate. PML is observed nearly exclusively in patients with severe immune deficiency, such as patients with acquired immune deficiency syndrome (AIDS) as well as patients receiving immunosuppressive therapies (e.g., steroids, cytostatics and antiproliferative agents, therapeutic antibodies, calcineurin inhibitors, anti-rejection drugs, and the like), such as patients with organ transplants, Hodgkin's lymphoma, multiple sclerosis, psoriasis, and other autoimmune diseases. Currently, there are no drugs to effectively inhibit or cure the viral infection; treatment relies primarily on reversing or relieving the immunodeficiency of the patient to slow or stop disease progression. Unfortunately, such strategies require suspending or halting therapy in immunosuppression patients, creating a dilemma that leaves these patients vulnerable to one of their two conditions. Thus, new therapies are needed to treat and prevent polyomavirus infections and/or polyomavirus-associated diseases

### SUMMARY

Provided herein are compositions and methods related to polyomavirus epitopes (e.g., epitopes listed in Tables 1, 2, 3, 4, 5 and/or 6) that are recognized by T lymphocytes (e.g., cytotoxic T lymphocytes (CTLs) and/or helper T lymphocytes) and that are useful in the prevention and/or treatment of a polyomavirus infection (e.g., a JCV infection), and/or cancer (e.g., a polyomavirus associated cancer, such as JCV associated cancer). In some embodiments, the compositions and methods relate to JCV epitopes (e.g., the epitopes listed in Tables 1, 2 and 3). In some embodiments, the compositions and methods relate to hybrid epitopes that incorporate sequence variations found within a viral strain and/or across related viral strains (e.g., the epitopes listed in Table 4).

In certain aspects, provided herein is a peptide (e.g., an isolated and/or recombinant polypeptide) comprising one or more epitopes from one or more JCV antigens ((e.g., epitopes from LTA, STA or VP1 viral antigens, such as the epitopes listed in Tables 1, 2 and 3) and/or one or more hybrid epitopes (e.g., the epitopes listed in Table 4). In some embodiments, the polypeptide comprises a plurality of such epitopes. In some embodiments, the polypeptide further comprises an intervening amino acid sequence between at least two of the plurality of epitopes. In some embodiments, the peptide is capable of eliciting an immune response upon administration to a subject (e.g., a mammalian subject, such as a human subject).

In some embodiments, the epitopes are selected to provide broad coverage of the human population. In some embodiments, the epitopes have HLA class I restrictions to HLA-A1, -A2, -A3, -A11, -A23, -A24, -A26, -A29, -A30, -B7, -B8, -B27, -B35, -B38, -B40, -B41, -B44, -B51, -B56, -B57 or -B58. In some embodiments, the epitopes have HLA class II restrictions to HLA-DP, -DM, -DOA, -DOB, -DQ, or -DR. In some embodiments, the epitopes have HLA class II restrictions to HLA-DRB or -DQB. In some embodiments, the peptide comprises, consists essentially of, or consists of epitope amino acid sequences set forth in SEQ ID NOS: 1 to 21. In some embodiments, provided herein is a pharmaceutical composition comprising a peptide provided herein.

In certain aspects, provided herein is a nucleic acid (e.g., an isolated nucleic acid) encoding a peptide disclosed herein. In some embodiments, provided herein is an expression construct comprising such a nucleic acid. In some embodiments, provided herein is a host cell comprising such an expression construct. In certain aspects provided herein is a method of producing an isolated peptide comprising expressing the isolated peptide in the host cell of provided herein and at least partly purifying the isolated peptide. In some embodiments, provided herein is a pharmaceutical composition comprising a nucleic acid provided herein.

In certain aspects, provided herein is a T lymphocyte (e.g., a an isolated T lymphocyte, a CD4+ T lymphocyte, a CD8+ T lymphocyte) comprising a T cell receptor (TCR) that specifically binds to an epitope described herein presented on an HLA (e.g., a class I HLA, a class II HLA). In certain embodiments, provided herein is a method of expanding BK virus-specific T lymphocytes for adoptive immunotherapy, including: (i) contacting one or more cells isolated from a subject, wherein the one or more cells comprise T lymphocytes, with an antigen presenting cell presenting an epitope provided herein; and (ii) culturing the one or more cells under conditions such that BK virus-specific T-lymphocytes are expanded from said one or more cells. In specific embodiments, culturing the one or more cells is performed in the presence of IL-2 and/or IL-21. In some embodiments, the cells are cultured in the presence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 ng/ml IL-2 and/or IL-21, In some embodiments, the cells are cultured in no more than 30, 35, 40, 45, 50, 60, 70, 80, 90 or 100 ng/ml IL-2 and/or IL-21. In some embodiments, the cells are cultured in 10-50, 20-40, 25-35 or about 30 ng/ml IL-2 and/or IL-21. In some embodiments, the cells are cultured in 30 ng/ml IL-2 and/or IL-21. In certain embodiments, compared to expansion in the absence of IL-2 and/or IL-21, expansion in the presence of IL-2 and/or IL-21 results in an increase in the ratio of absolute number of polyomavirus-specific CD8 T cells to the absolute number of polyomavirus-specific CD4 T cells in the expanded population of T lymphocytes.

In certain embodiments, provided herein is a method of treating or preventing a polyomavirus infection (e.g., a JCV infection) and/or treating a polyomavirus-associated cancer (e.g., a JCV-associated cancer) and/or inducing a T-lymphocyte immune response in a subject comprising administering to the subject a peptide, nucleic acid, T cell or pharmaceutical composition provided herein. In some embodiments, the subject is a mammal. In some embodiments, the subject is a human. In some embodiments, the subject is immunocompromised.

In certain aspects, provided herein is a method of detecting a JC virus infection in a subject, the method comprising detecting the presence of JCV-specific T lymphocytes by contacting T lymphocytes isolated from the subject with the isolated peptide provided herein. In some embodiments, the method further comprising treating the JC virus infection in the subject according to a method described herein. In some embodiments, the subject is a mammal. In some embodiments, the subject is a human. In some embodiments, the subject is immunocompromised.

In certain aspects, provided herein are methods of treating a cancer in a subject (e.g., a polyomavirus-associated cancer, such as a JCV- -associated cancer). In some embodiments, the method comprises administering to the subject a pharmaceutical composition comprising cytotoxic T cells (CTLs) comprising T cell receptors (TCRs) that recognize one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4. In some embodiments, the subject expresses a human leukocyte antigen (HLA) to which the one or more epitopes is restricted. In some embodiments, the CTLs are autologous to the subject. In some embodiments, the CTLs are not autologous to the subject. In some embodiments, the CTLs are obtained from a CTL library or bank. In some embodiments, the method comprises administering to the subject a vaccine composition comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4. In some embodiments, the method comprises administering to the subject a pharmaceutical composition antigen presenting cells (APCs) presenting one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4. In some embodiments, the subject expresses a human leukocyte antigen (HLA) to which the one or more epitopes is restricted.

In certain aspects, provided herein are methods of treating a polyomavirus infection (e.g. a JCV infection) in a subject. In some embodiments, the subject is immunocompromised. In some embodiments, the method comprises administering to the subject a pharmaceutical composition comprising CTLs comprising TCRs that recognize one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4. In some embodiments, the subject expresses a HLA to which the one or more epitopes is restricted. In some embodiments, the CTLs are autologous to the subject. In some embodiments, the CTLs are not autologous to the subject. In some embodiments, the CTLs are obtained from a CTL library or bank. In some embodiments, the method comprises administering to the subject a vaccine composition comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4. In some embodiments, the method comprises administering to the subject a pharmaceutical composition antigen presenting cells (APCs) presenting one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4. In some embodiments, the subject expresses human leukocyte antigens (HLA) to which the one or more epitopes is restricted.

In some aspects, provided herein is a population of CTLs comprising T cell receptors (TCRs) that recognize one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4.

In some aspects, provided herein is a population of APCs presenting one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4. In some embodiments, the APCs comprise B cells, antigen-presenting T cells, dendritic cells and/or artificial antigen-presenting cells, such as aK562 cells. In some aspects, the antigen-presenting cells (e.g., aK562 cells) express CD80, CD83, 41BB-L, and/or CD86. In some embodiments, provided herein are methods of treating or preventing cancer (e.g., a polyomavirus associated cancer, such as a JCV associated cancer) and/or a polyomavirus (e.g., JCV) infection in a subject comprising administering the APCs described herein to a subject.

In some aspects, provided herein is a polypeptide comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4. In certain aspects, provided herein is a nucleic acid molecule (e.g., a DNA molecule or an RNA molecule) encoding a polypeptide comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4. In some embodiments, the nucleic acid molecule is a vector (e.g., an adenoviral vector). In some embodiments, provided herein are vaccine compositions comprising a polypeptide and/or a nucleic acid molecule described herein.

In some embodiments, provided herein are methods of generating, activating and/or inducing proliferation of polyomavirus-specific CTLs (e.g., JCV-specific CTLs) comprising contacting CTLs with APCs that present one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4. In some embodiments, the CTLs are contacted with APCs in vitro. In some embodiments, the APCs comprise B cells, antigen-presenting T cells, dendritic cells and/or artificial antigen-presenting cells, such as aK562 cells. In some aspects, the antigen-presenting cells (e.g., aK562 cells) express CD80, CD83, 41BB-L, and/or CD86. In some embodiments, the CTLs are contacted to the APCs in the presence of one or more cytokines.

In some embodiments, provided herein are methods of generating APCs that present epitopes provided herein comprising contacting APCs with a polypeptide comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4 and/or a nucleic acid encoding a polypeptide comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2, Table 3 and/or Table 4. In some embodiments, the APCs express HLA to which the one or more epitopes is restricted.

In some embodiments, the one or more epitopes comprise an epitope shared by two or more polyomaviruses. In some embodiments, the shared epitope comprises a region of sequence homology between the at least two polyomaviruses, and the region of sequence homology is at least 3, 4, 5, 6 or 7 amino acids across the full length of the epitope sequence. In some embodiments, the two polyomaviruses are BKV and JCV. In some embodiments, the at least three amino acids are LLL.

In other aspects, provided herein is a method of identifying a subject suitable for a method of treatment provided herein (e.g., administration of CTLs, APCs, or vaccine compositions provided herein) comprising isolating a sample from the subject (e.g., a blood or tumor sample) and detecting the presence of an epitope provided herein, or a nucleic acid encoding an epitope provided herein. In certain embodiment, the subject is identified as suitable for a method of treatment provided herein if the subject expresses an HLA to which one or more of the epitopes described herein are restricted. In some embodiments, the subject identified as being suitable for a method of treatment provided herein is treated using the method of treatment.

### BRIEF DESCRIPTION OF THE DRAWINGS

FIG. 1 shows T cell response to JCV antigens. Briefly, PBMCs from 17 healthy subjects were stimulated with JCV overlapping peptide pools (OPPs) and T cells expanded for 14 days in the presence of IL-2. The cells were assayed for the expression of IFN-γ on the 14th day upon stimulation with respective peptide pools using flow cytometry. The compiled data of all 17 donors is represented in the graphs; (A) showing the response of BKV specific CD8 T cells and (B) the response of CD4⁺ T cells, after stimulation with respective BKV OPPs.

FIGS. 2A and 2B depict representative data demonstrating the identification of T cell determinants using a two dimensional peptide matrix. JCV-specific T cells expanded in vitro using the OPPs were further characterized by identifying specific T cell determinants. FIG. 2A shows individual overlapping peptides were used to make subpools (24 pools; LTA1-LTA24) and the T cell response for each pool was measured by intracellular cytokine-staining (ICS) IFN-y assay as represented in the bar graph. The response using the subpools were overlayed on a two dimensional matrix, thus showing the common individual peptides among the pools. FIG. 2B shows FACs plots that demonstrate the T cell response for each individual peptide (P29, P30, and P32) when used in the IFN-γ ICS assay thereby confirming the peptides responsible for eliciting JCV-specific T cell response. Figure discloses SEQ ID NOS 2-4, respectively, in order of appearance.

FIG. 3 shows representative data of the HLA class II restriction analysis for epitopes mapped from JCV-LT antigen. Specifically, the HLA class II-restriction of VDLHAFLSQAVFSNR (SEQ ID NO: 2) (LT29), FLSQAVFSNRTVASF (SEQ ID NO: 3) (LT30) and TVASFAVYTTKEKAQ (SEQ ID NO: 4) (LT32) peptides as HLA DRB1*10:01 is shown. Briefly, a panel of lymphoblastoid cells (LCLs) matching one single allele of the HLA type of the donor were chosen and loaded with respective peptide for 1 hour. The loaded LCLs were then used as stimulator cells in an IFN-γ ICS assay. The peptide, when presented by the MHC, elicits an IFN-γ response in JCV-specific T cells.

FIG. 4 demonstrates T cell cross reactivity between BKV and JCV epitopes. ICS FACS plots show the expression of IFN-γ in T cells expanded with either BKV epitope (SSGTQQWRGLARYFK (SEQ ID NO: 33)) or JCV epitope (RSGSQQWRGLSRYFK (SEQ ID NO: 12)). These primed T cells were stimulated using both BKV and JCV peptide showing that the T cells expanded with either of these epitopes recognize both BKV and JCV peptide sequences.

FIG. 5 illustrates JCV-specific T cell expansion from healthy subjects. The frequency of CD4⁺ T cells expressing IFN-γ was assessed and the response of each individual subject is shown in the graph.

FIG. 6 shows the polyfunctionality of JCV-specific T cells expanded using the pooled peptides. FIG. 6A shows a representative FACs dot plots show the expression of individual effector molecules in CD4⁺ T cells upon re-stimulation with JCV peptide pool. FIGS. 6B and 6C show the polyfunctionality of JCV-specific T cells expressing multiple cytokines.

FIG. 7 shows the transcription factor and effector profile of JCV-specific T cells grown in vitro.

### DETAILED DESCRIPTION

General

Provided herein are compositions and methods related to polyomavirus epitopes (e.g., epitopes listed in Tables 1, 2, 3 and/or 4) that are recognized by T lymphocytes (e.g., cytotoxic (CD8⁺) T lymphocytes (CTLs) and/or helper (CD4⁺) T lymphocytes) and that are useful in the prevention and/or treatment of a polyomavirus infection (e.g., a JCV infection), and/or cancer (e.g., a polyomavirus associated cancer, such as a JCV associated cancer). In some embodiments, the compositions and methods provided herein relate to JCV epitopes (e.g., the epitopes listed in Tables 1, 2 and 3). In some embodiments, the compositions and methods relate to hybrid epitopes that encompass variations found within or across BKV and JCV epitopes (e.g., the epitopes listed in Table 4).

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, peptide described herein, an antigen presenting cell provided herein and/or a CTL provided herein.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of any of the foregoing.

The term “binding” or “interacting” refers to an association, which may be a stable association, between two molecules, e.g., between a TCR and a peptide/HLA, due to, for example, electrostatic, hydrophobic, ionic and/or hydrogen-bond interactions under physiological conditions. A TCR “recognizes” a T cell epitope that it is capable of binding to when the epitope is presented on an appropriate HLA.

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

As used herein, the term “cancer” includes, but is not limited to, solid tumors and blood borne tumors. The term cancer includes diseases of the skin, tissues, organs, bone, cartilage, blood and vessels. The term “cancer” further encompasses primary and metastatic cancers.

The term “homologous” as used herein, refers to sequence similarity (e.g., a nucleic acid or amino acid sequence) between two regions of the same sequence strand or between regions of two different sequence strands. The term “homologous” may also be used to refer to sequence similarity between two regions of the same sequence strand or between regions of two different sequence strands. For example, when an amino acid residue position in both regions is occupied by the same amino acid residue, then the regions are homologous at that position. A first region is homologous to a second region if at least one nucleotide residue position of each region is occupied by the same residue. Homology between two regions is expressed in terms of the proportion of nucleotide or amino acid residue positions of the two regions that are occupied by the same nucleotide or amino acid residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably, at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “isolated” refers to material that has been removed from its natural state or otherwise been subjected to human manipulation. Isolated material may be substantially or essentially free from components that normally accompany it in its natural state, or may be manipulated so as to be in an artificial state together with components that normally accompany it in its natural state.

The term “peptide” refers to two or more amino acids linked together by peptide bonds or modified peptide bonds. The term “peptide”, “polypeptide” and “protein” in usage herein may be used interchangeably. In certain embodiments, the peptide is prepared from recombinant DNA or RNA, or of synthetic origin, or some combination thereof, which (1) is not associated with peptides that it are normally found with in nature, (2) is isolated from the cell in which it normally occurs, (3) is isolated free of other peptides from the same cellular source, (4) is expressed by a cell from a different species, or (5) does not occur in nature.

The term “epitope” means a peptide determinant capable of specific binding to an antibody or TCR. Epitopes usually consist of chemically active surface groupings of molecules such as amino acids or sugar side chains. Certain epitopes can be defined by a particular sequence of amino acids to which an antibody is capable of binding.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, “specific binding” refers to the ability of an antibody to bind to a predetermined antigen or the ability of a peptide to bind to its predetermined binding partner. Typically, an antibody or peptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein).

As used herein, the term “subject” means a human or non-human animal selected for treatment or therapy.

The phrases “therapeutically-effective amount” and “effective amount” as used herein means the amount of an agent which, when administered to a subject, elicits adequate therapeutic response in the subject to provide beneficial outcome in the subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that may or may not be able to replicate autonomously or integrate into a chromosome of a host cell.

### Epitopes

In certain embodiments provided herein are methods and compositions related to polyomavirus epitopes, such as JCV epitopes, that are recognized by immune effector cells (e.g., cytotoxic T cells/CTLs) when presented on an HLA. In certain embodiments, the epitopes described herein are useful in the prevention and/or treatment of a polyomavirus infection (e.g., a JCV viral infection) and/or cancer (e.g., a JVC-associated cancer expressing an epitope provided herein) and/or for the generation of pharmaceutical agents and compositions thereof (e.g., sensitized immune effector cells and/or APCs) that are useful in the prevention and/or treatment of a polyomavirus infection (e.g., JCV viral infections) and/or cancer (e.g., a polyomavirus associated cancer expressing an epitope provided herein). In certain embodiments, the epitope is a JCV epitope listed in Table 1, Table 2 and/or Table 3. In some embodiments, the epitope is a hybrid epitope comprising amino acids from both a BKV epitope and a homologous JCV epitope and/or amino acid variants found within different BKV or JCV strains. Exemplary hybrid epitopes are listed in Table 4. In some embodiments, the compositions and methods described herein further relate to epitopes from addition viruses, such as EBV, CMV, or ADV. In some embodiments, the epitopes are HLA class I-restricted T cell epitopes. In other embodiments, the epitopes are HLA class II-restricted T cell epitopes.

TABLE 1
Exemplary JCVHLA-restricted T cell epitopes

| JCV epitope peptide | JCV | | SEQ |
|---|---|---|---|
| sequence | Antigen | HLA Restriction | ID NO.: |
| IDQFMVVFEDVKGTG | LTA | DRB1*14:04/DQB1*05:03 | 1 |
| VDLHAFLSQAVFSNR | LTA | DRB1*10:01 | 2 |
| FLSQAVFSNRTVASF | LTA | | 3 |
| TVASFAVYTTKEKAQ | LTA | | 4 |
| ERLNFELGVGIDQFM | LTA | DRB1*15:01 | 5 |
| TCGNILMWEAVTLKT | VP1 | | 6 |
| RYWLFKGPIDSGKTT | LTA | | 7 |
| MTREEMLVERFNFLL | LTA | DRB1*04:01 & DRB1*03:01 | 8 |
| EQYMAGVAWIHCLLP | LTA | | 9 |
| RKAYLKKCKELHPDK | STA | DRB1*13:01 | 10 |
| GGHNILFFLTPHRHR | LTA | DRB1*16:01 | 11 |
| RSGSQQWRGLSRYFK | VP1 | DRB1*11:01 | 12 |
| DPDMMRYVDKYGQLQ | VP1 | DRB1*04:02 | 13 |
| MDKVLNREESMELMD | LTA | DRB1*01:01 | 14 |
| SITEVECFLTPEMGD | VP1 | DRB1*01:01 & DQB1*05:01 | 15 |
| SKNQKSICQQAVDTV | LTA | DRB1*04:01 & DQB1*02:02 | 16 |
| SICQQAVDTVAAKQR | LTA | | 17 |
| RNRKFLRSSPLVWID | LTA | DRB1*15:01 | 18 |
| LRSSPLVWIDCYCFD | LTA | | 19 |
| KMKRMNFLYKKMEQG | LTA | | 20 |
| NFLYKKMEQGVKVAH | LTA | | 21 |

TABLE 2
List ofJCV epitopes and homologous BKT epitope peptide sequences

| | | Homologous BKV | |
|---|---|---|---|
| JCV epitope peptide | SEQ ID | epitope peptide | SEQ ID |
| sequences | NO.: | sequences | NO.: |
| IDQFMVVFEDVKGTG | 1 | IDQYMVVFEDVKGTG | 22 |
| VDLHAFLSQAVFSNR | 2 | SDLHQFLSQAVFSNR | 23 |
| FLSQAVFSNRTVASF | 3 | FLSQAVFSNRTLACF | 24 |
| TVASFAVYTTKEKAQ | 4 | TLACFAVYTTKEKAQ | 25 |
| ERLNFELGVGIDQFM | 5 | ERLTFELGVAIDQYM | 26 |
| TCGNILMWEAVTLKT | 6 | TCGNLLMWEAVTVKT | 27 |
| RYWLFKGPIDSGKTT | 7 | RYWLFKGPIDSGKTT | 28 |
| MTREEMLVERFNFLL | 8 | MTREEMLTERFNHIL | 29 |
| EQYMAGVAWIHCLLP | 9 | EQYMAGVAWLHCLLP | 30 |
| RKAYLKKCKELHPDK | 10 | RKAYLRKCKEFHPDK | 31 |
| GGHNILFFLTPHRHR | 11 | AGHNIIFFLTPHRHR | 32 |
| RSGSQQWRGLSRYFK | 12 | SSGTQQWRGLARYFK | 33 |
| DPDMMRYVDKYGQLQ | 13 | DPDMIRYIDRQGQLQ | 34 |
| MDKVLNREESMELMD | 14 | MDKVLNREESMELMD | 35 |
| SITEVECFLTPEMGD | 15 | AITEVECFLNPEMGD | 36 |
| SKNQKSICQQAVDTV | 16 | SKNQKSICQQAVDTV | 37 |
| SICQQAVDTVAAKQR | 17 | SICQQAVDTVLAKKR | 38 |
| RNRKFLRSSPLVWID | 18 | LNRKFLRKEPLVWID | 39 |
| LRSSPLVWIDCYCFD | 19 | LRKEPLVWIDCYCID | 40 |
| KMKRMNFLYKKMEQG | 20 | KMKRMNTLYKKMEQD | 41 |
| NFLYKKMEQGVKVAH | 21 | NTLYKKMEQDVKVAH | 42 |

TABLE 3
Exemplary epitope sequences from JCV homologous to EKE epitope sequences

| Epitope* | Antigen | HLA Restriction | SEQ ID NO.: |
|---|---|---|---|
| KS(Q/R)HSTPP(K/R)K | LTA | A*11 | 43 |
| AVDTVAAKQ | LTA | A*11 | 44 |
| CYCFDCFRQ | STA | A*24 | 45 |
| IPVMRKAYL | LTA/STA | B*07/B*08 | 46 |
| FPPNSDTLY | STA | B*35 | 47 |
| FLYCKEWPN | STA | B*35 | 48 |
| SPLV(W/R)IDCY | STA | B*35 | 49 |
| VHCPCLMCML | STA | B*39 | 50 |
| NREESMELMDLL | LTA/STA | B*40 | 51 |
| MELMDLLGL | LTA/STA | B*40 | 52 |
| FFSVGGEALEL | VP1 | B*40 | 53 |
| YCFDCFRQW | STA | B*57 | 54 |

| | | | |
|---|---|---|---|
| TPHRHRVSA | LTA | B*56 | 55 |
| LLMGMYLDF | LTA | A*29 | 56 |
| VFLLMGMYLDF | LTA | A*23 | 57 |
| VE(E/G)SIQGGL | LTA | B*40 | 58 |
| TEV(I/L)GVTLMN | VP1 | B*40 | 59 |
| ARIPLPNLN | VP1 | B*27 | 60 |
| VKNPYPISFLL | VP1 | Cw*07 | 61 |
| LPGDPDMMRYVDKYG | VP1 | HLA A24/A29/B7/B39 | 62 |
| LEVKTGVDSITEVEC | VP1 | HLA A24/A29/B7/B39 | 63 |
| DVCGMFTNRSGSQQW | VP1 | HLA A24/A29/B7/B39 | 64 |
| DAQVEEVRVFEGTEE | VP1 | HLA A24/A29/B7/B39 | 65 |
| QAVDTVAAKQ | LTA | A*11 | 66 |
| ML(V/M)(E/Q/G)RFNFLL | LTA | A*02 | 67 |
| LLLIWFRPV | LTA | A*02:01 | 68 |
| S(I/V)TEVECFL | VP1 | A*02:01 | 69 |
| RLDLEISMY | LTA | A*01 | 70 |
| SV(K/R)VNLERKH | LTA | A*03 | 71 |
| AYLK(K/R)CKEL | LTA | A*24 | 72 |
| (N)LLMWEAVTV | VP1 | A*02 | 73 |
| GSQQWRGLSRYFKVQ | VP1 | DRB1*11/8 | 74 |
| RGLSRYFKVQLRKRR | LTA | DRB1*11/8 | 75 |
| RKAYLKKCKELHPDK | LTA | DRB1*13 | 76 |
| WDEDLFCHEEMFASD | LTA | DQB5*01 | 77 |
| CFRQWFGCDLTQEAL | LTA/STA | DRB1*03/04 | 78 |
| GGDEDKMKRMNFLYK | LTA | DRB1*13 | 79 |
| KMKRMNFLYKKMEQG | VP1 | DRB1*13 | 80 |
| LNIPKKRYWLFKGPIDSGKT | VP1 | DRB1*15 | 81 |
| KRYWLFKGPIDSGKT | VP1 | DRB1*15 | 82 |
| VGPLCKGDNLYLSAV | VP1 | DRB1*01 | 83 |
| AYLDKNKAYPVECWV | VP1 | ND** | 84 |
| DMMRYVDRYGQLQTK | VP1 | ND** | 85 |
| SQHSTPPKK | LTA | A*11 | 86 |
| FPPNSDTLYC | STA | B*35 | 87 |
| LLIKGGVEV | ND* | ND* | 88 |

*Amino acid residues which are variant from BKV epitopes are bolded and underlined.
**Not defined

TABLE 4
Exemplary epitope sequences from JCV/BKV hybrid epitope sequences

| | | HLA | SEQ ID |
|---|---|---|---|
| Epitope* | Antigen | Restriction | NO.: |
| (D/K)S(Q/K)HSTPP(K/R/KK) | LTA | A*11 | 89 |
| AVDTV(L/A)AK(K/Q) | LTA | A*11 | 90 |
| CYC(I/F)DCF(T/R)Q | STA | A*24 | 91 |
| (L/I)P(L/V)MRKAYL | LTA/STA | B*07/B*08 | 92 |
| FP(L/P)(P/N)(P/S)DTLY | STA | B*35 | 93 |
| (F/T)LYCKEWP(I/N) | STA | B*35 | 94 |
| (E/S)PL(VWI/VWK/VRI/GWI)DCY | STA | B*35 | 95 |
| VHCPC(M/L)(L/M)C(M/Q)L | STA | B*39 | 96 |
| YC(I/F)DCF(T/R)(Q/E)W | STA | B*57 | 97 |
| LL(L/M)GMYL(E/D)F | LTA | A*29 | 98 |
| V(F/L)LLMGMYL(E/D)F | LTA | A*23 | 99 |
| (I/V)E(E/G)SI(Q/H)GGL | LTA | B*40 | 100 |
| TEV(I/M/L)G(I/V)T(S/L)M(L/N) | VP1 | B*40 | 101 |
| LPGDPDM(I/M)RY(I/V)D(R/K)(Q/Y)G | VP1 | HLA | 102 |
| | | A24/A29/B7/ | |
| | | B39 | |
| D(I/V)CG(L/M)F(T/I)N(S/R)SG(T/S) | VP1 | HLA | 103 |
| QQW | | A24/A29/B7/ | |
| | | B39 | |
| QAVDTV(L/A)AK(K/Q) | LTA | A*11 | 104 |
| ML(T/V/M)(E/D/Q/E)RFN(H/F)(I/L)L | LTA | A*02 | 105 |
| (AI/SI/SV)T(E/Q)VECFL | VP1 | A*02:01 | 106 |
| (R/K)LD(S/L)EISMY | LTA | A*01 | 107 |
| SV(K/R)VNLE(E/R)KH | LTA | A*03 | 108 |
| AYL(R/K)KCKE(F/L) | LTA | A*24 | 109 |
| (N)(I/V)MWEAVT(L/V) | VP1 | A*02 | 110 |
| G(T/S)QQWRGL(A/S)RYFK(I/V)(R/Q) | VP1 | DRB1*11/8 | 111 |
| RGL(A/S)RYFK(I/V)(R/Q)LRKR(S/R) | LTA | DRB1*11/8 | 112 |
| RKAYL(RR/RK/KK)CKE(F/L)HPDK | LTA | DRB1*13 | 113 |
| WDEDLFCHE(D/E)MFASD | LTA | DQB5*01 | 114 |
| CF(T/R)QWFG(L/C)DLT(E/Q)E(T/A)L | LTA/STA | DRB1*03/04 | 115 |
| GGDEDKMKRMN(T/F)LYK | LTA | DRB1*13 | 116 |
| KMKRMN(T/F)LYKKMEQ(D/G) | VP1 | DRB1*13 | 117 |
| (F/L)N(V/I)PK(R/K)RYWLFKGPIDSGKT | VP1 | DRB1*15 | 118 |
| (R/K)RYWLFKGPIDSGKT | VP1 | DRB1*15 | 119 |
| VGPLCK(A/G)D(S/N)LYLSAV | VP1 | DRB1*01 | 120 |
| AYLDKN(N/K)AYPVECW(I/V) | VP1 | ND** | 121 |
| DM(I/M)RY(I/V)DR(Q/G)GQLQTK | VP1 | ND** | 122 |

**Not defined

In some embodiments, provided herein are peptides (e.g., polypeptides) comprising one or more of the epitopes from Table 1, Table 2, Table 3 and/or Table 4. In some embodiments, the peptides disclosed herein are full-length viral proteins (e.g., full-length BKV and/or JCV proteins). In some embodiments, the peptide is not a full-length viral protein (e.g., not a full-length BKV and/or JCV protein). In some embodiments, the peptides disclosed herein comprise BKV and JCV epitopes with sequence homology (e.g., epitopes listed in Tables 2, 3 and 4). In some embodiments, the peptides disclosed herein comprise less than 100, 90, 80, 70, 60, 50, 40, 30, 25, 20, 15 or 10 contiguous amino acids of a viral protein. In some embodiments, the peptides disclosed herein comprise two or more of the epitopes listed in Table 1, Table 2, Table 3 and/or Table 4. For example, in some embodiments, the peptides disclosed herein comprise two or more of the epitopes listed in Table 1, Table 2, Table 3 and/or Table 4 connected by polypeptide linkers. In some embodiments, the peptide provided herein comprises at least 1, 2, 3, 4, 5, 6, 7, 8, 9, 10, 11, 12, 13, 14, 15, 16, 17, 18, 19, 20, 21, 22, 23, 24, 25, 26, or 27 epitopes (e.g., at least 1, 2, 3, 4, 5, 6, 7, 8, 9, 10, 11, 12, 13, 14, 15, 16, 17, 18, 19, 20, 21, 22, 23, 24, 25, 26, or 27 of the epitopes listed in Table 1, Table 2, Table 3 and/or Table 4). In preferred embodiments, the peptides disclosed herein comprise the JCV epitopes set forth in Table 1, i.e., any one of the JCV epitopes set forth in SEQ ID Nos: 1-21, or any combination thereof. For example, the peptide may comprise at least 1, 2, 3, 4, 5, 6, 7, 8, 9, 10, 11, 12, 13, 14, 15, 16, 17, 18, 19, 20 or all 21 of the epitopes encoded by the amino acid sequences set forth in SEQ ID Nos: 1-21.

In certain aspects, provided herein are polypeptides (e.g., isolated polypeptides and/or recombinant polypeptides) comprising a plurality of epitopes from BKV or JCV antigens (e.g., epitopes from large T-antigen (LTA), small T-antigen (STA) or major capsid protein VP1 viral antigens, such as those epitopes listed in Tables 1, 2, 3 or 4), preferably the epitopes set forth in Table 1. More preferably, the polypeptides disclosed herein comprise any one of the JCV epitopes set forth in SEQ ID Nos: 1-21, or any combination thereof. In some such embodiments, the polypeptide further comprises an intervening amino acid sequence between at least two of the plurality of epitopes. In some embodiments, the intervening amino acids or amino acid sequences are proteasome liberation amino acids or amino acid sequences. Non-limiting examples of proteasome liberation amino acids or amino acid sequences are or comprise AD, K or R. In some embodiments, the intervening amino acids or amino acid sequence are TAP recognition motifs. Typically, TAP recognition motifs may conform to the following formula: (R/N:I/Q:W/Y), where n is any integer >1. Non-limiting examples of TAP recognition motifs include RIW, RQW, NIW and NQY. In some embodiments, the epitopes provided herein are linked or joined by the proteasome liberation amino acid sequence and, optionally, the TAP recognition motif at the carboxyl terminus of each epitope. In some such embodiments, the polypeptide comprises, or consists essentially of, each of the epitopes encoded by the amino acid sequences set forth in SEQ ID Nos: 1-21.

In some embodiments, the polypeptides provided herein further comprise epitopes from and at least one additional virus (e.g., Epstein Barr virus (EBV), cytomegalovirus (CMV), and/or adenovirus (ADV)). In some embodiments the peptides comprise epitopes two or more viruses. In some embodiments the peptides comprise epitopes three or more viruses. In some embodiments the peptides comprise epitopes four or more viruses. In some embodiments the peptides comprise epitopes five or more viruses. For example, in some embodiments the peptides comprise sequences from at least two, three, four or five of JCV, BKV, EBV, CMV and/or ADV.

In some embodiments, provided herein is a polyepitope polypeptide (i.e., a single chain of amino acid residues comprising multiple T cell epitopes not linked in nature) comprising two or more of the epitopes described herein. In some embodiments, the T cell epitopes in the polypeptide are connected via an amino acid linker. In some embodiments, the T cell epitopes in the polypeptide are directly linked without intervening amino acids. Examples of polyepitope polypeptides, methods of generating polyepitope polypeptides, and vectors encoding polyepitope polypeptides can be found in Dasari et al., Molecular Therapy—Methods & Clinical Development (2016) 3, 16058, which is hereby incorporated by reference in its entirety.

In certain aspects, provided herein are pools of immunogenic peptides comprising HLA class I and class II-restricted polyomavirus peptide epitopes (e.g., epitopes listed in Tables 1, 2, 3, 4, 5 and/or 6) capable of inducing proliferation of peptide-specific T cells. In some embodiments, the pool of immunogenic peptides comprises at least 1, 2, 3, 4, 5, 6, 7, 8, 9, 10, 11, 12, 13, 14, 15, 16, 17, 18, 19, 20, 21, 22, 23, 24, 25, 26, or 27 epitopes (e.g., at least 1, 2, 3, 4, 5, 6, 7, 8, 9, 10, 11, 12, 13, 14, 15, 16, 17, 18, 19, 20, 21, 22, 23, 24, 25, 26, or 27 of the epitopes listed in Table 1, Table 2, Table 3 and/or Table 4), or combinations thereof. In preferred embodiments, the peptide pool comprises at least one JCV epitope set forth in Table 1, i.e., any one of the JCV epitopes set forth in SEQ ID Nos: 1-21, or any combination thereof. For example, the pool of immunogenic peptides may comprise at least 1, 2, 3, 4, 5, 6, 7, 8, 9, 10, 11, 12, 13, 14, 15, 16, 17, 18, 19, 20 or all 21 of the epitopes encoded by the amino acid sequences set forth in SEQ ID Nos: 1-21. Most preferably, such peptide pools comprise each of the JCV peptide epitope amino acid sequences set forth in in SEQ ID Nos: 1-21. The immunogenic peptides, and pools thereof, are capable of inducing proliferation of peptide-specific T cells (e.g., peptide-specific cytotoxic T-cells and/or CD4⁺ T cells).

In some embodiments, the compositions and methods provided herein comprise or relate to naturally occurring variants of the epitopes listed in Tables 1, 2, and/or 3. For example, in some embodiments, provided herein is a polyepitope polypeptide that comprises two or more (e.g., at least 3, 4, 5, 6, 7, 8, 9 or 10) naturally occurring variants of an epitope listed in Table 1, Table 2 and/or Table 3.

In some embodiments, the sequence of the epitopes provided herein have a sequence disclosed herein except for 1 or more (e.g., 1, 2, 3, 4, 5, 6, 7, 8, 9, 10 or more) conservative sequence modifications. As used herein, the term “conservative sequence modifications” is intended to refer to amino acid modifications that do not significantly affect or alter the interaction between a TCR and a peptide containing the amino acid sequence presented on an HLA. Such conservative modifications include amino acid substitutions, additions (e.g., additions of amino acids to the N or C terminus of the peptide) and deletions (e.g., deletions of amino acids from the N or C terminus of the peptide). Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues of the peptides described herein can be replaced with other amino acid residues from the same side chain family and the altered peptide can be tested for retention of TCR binding (e.g., antigenicity) using methods known in the art. Modifications can be introduced into an antibody by standard techniques known in the art, such as site-directed mutagenesis and PCR-mediated mutagenesis.

In some embodiments, the peptides (e.g., polypeptides) described herein are immunogenic and are capable of eliciting an immune response upon administration to a subject (e.g., a mammalian subject, such as a human subject). In further embodiments, the peptides (e.g., polypeptides) described herein are capable of eliciting an immune response following endogenous or exogenous processing and/or presentation of the peptides by immune cells (e.g., immune cells of the subject and/or immune cells from a donor such as those immune cells comprising allogeneic PBMCs.

In some aspects, provided herein are cells that present one or more of the peptides described herein (e.g., a peptide comprising at least one epitope listed in Table 1, Table 2, Table 3 and/or Table 4). In some embodiments, the cell is a mammalian cell. In some embodiments the cell is an antigen-presenting cell (APC) (e.g., an antigen-presenting T-cell, a dendritic cell, a B cell, a macrophage or am artificial antigen-presenting cell, such as aK562 cell). A cell presenting a peptide described herein can be produced by standard techniques known in the art. For example, a cell may be pulsed to encourage peptide uptake. In some embodiments, the cells are transfected with a nucleic acid encoding a peptide provided herein. In some aspects, provided herein are methods of producing antigen-presenting cells (APCs), comprising pulsing a cell with the peptides described herein. Exemplary examples of producing antigen-presenting cells can be found in WO2013088114, hereby incorporated in its entirety.

The peptides provided herein can be isolated from cells or tissue sources by an appropriate purification scheme using standard protein purification techniques, can be produced by recombinant DNA techniques, and/or can be chemically synthesized using standard peptide synthesis techniques. The peptides described herein can be produced in prokaryotic or eukaryotic host cells by expression of nucleotides encoding a peptide(s) of the present invention. Alternatively, such peptides can be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N. Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### Nucleic Acid Molecules

Provided herein are nucleic acid molecules that encode the epitopes and peptides described herein. The nucleic acids may be present, for example, in whole cells, in a cell lysate, or in a partially purified or substantially pure form. A nucleic acid molecule described herein can be isolated using standard molecular biology techniques and the sequence information provided herein. For example, oligonucleotides corresponding to the nucleotide sequence of one or more of the epitopes listed in Tables 1, 2, 3, or 4 can be prepared by standard synthetic techniques, i.e., using an automated DNA synthesizer.

In some embodiments, provided herein are vectors (e.g., a viral vector, such as an adenovirus based expression vector) that contain the nucleic acid molecules described herein. A viral vector may contain additional DNA segments may be ligated into the viral genome. Certain vectors are capable of autonomous replication in a host cell into which they are introduced (e.g., bacterial vectors having a bacterial origin of replication, episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”). In some embodiments, provided herein are nucleic acids operable linked to one or more regulatory sequences (e.g., a promoter) in an expression vector. In some embodiments the cell transcribes the nucleic acid provided herein and thereby expresses an antibody, antigen binding fragment thereof, or peptide described herein. The nucleic acid molecule can be integrated into the genome of the cell or it can be extrachromosomal.

In some embodiments, the nucleic acid vectors or recombinant adenoviruses provided herein encode one or more epitopes listed in Tables 1, 2, 3, and/or 4. For example, the nucleic acid vectors or recombinant adenoviruses may consist of one or more epitopes from the same table (e.g., one or more epitopes from Table 1, one or more epitopes from Table 2, one or more epitopes from Table 3, or one or more epitopes from Table 4). Or, the nucleic acid vectors or recombinant adenoviruses may consist of one or more epitopes from the same table (e.g., Table 1), and one or more epitopes from a different table (e.g., Table 2). In some embodiments, the nucleic acid vectors or recombinant adenoviruses provided herein encode for no more than 20, 19, 18, 17, 16, 15, 14, 13, 12, 11, 10, 9, 8, 7, 6, 5, 4, 3, 2 or 1 amino acids in addition to the epitopes listed in Tables 1, 2, 3, and/or 4.

In some embodiments, the nucleic acid vectors comprise nucleic acid sequences that have undergone codon optimization. In such embodiments, a coding sequence is constructed by varying the codons in each nucleic acid used to assemble the coding sequence. In general, a method to identify a nucleotide sequence that optimizes codon usages for production of a peptide comprises at least the following steps (a) through (e). In step (a), oligomers are provided encoding portions of the polypeptide containing degenerate forms of the codon for an amino acid encoded in the portions, with the oligomers extended to provide flanking coding sequences with overlapping sequences. In step (b), the oligomers are treated to effect assembly of the coding sequence for the peptide. The reassembled peptide is included in an expression system that is operably linked to control sequences to effect its expression. In step (c), the expression system is transfected into a culture of compatible host cells. In step (d), the colonies obtained from the transformed host cells are tested for levels of production of the polypeptide. In step (e), at least one colony with the highest or a satisfactory production of the polypeptide is obtained from the expression system. The sequence of the portion of the expression system that encodes the protein is determined. Further description of codon optimization is provided in U.S. Patent Publication number US2010/035768, which is incorporated by reference in its entirety.

### Antigen-Presenting Cells

In some aspects, provided herein are APCs that present (e.g., on HLA) one or more T cell epitopes provided herein (e.g., one or more T cell epitopes listed in Table 1, Table 2, Table 3 and/or Table 4). In some embodiments, the HLA is a class I HLA. In some embodiments, the HLA is a class II HLA. In some embodiments, the class I HLA has an α chain polypeptide that is HLA-A, HLA-B, HLA-C, HLA-E, HLA-F, HLA-g, HLA-K or HLA-L. In some embodiment, the class II HLA has an a chain polypeptide that is HLA-DMA, HLA-DOA, HLA-DPA, HLA-DQA or HLA-DRA. In some embodiments, the class II MHLA has a β chain polypeptide that is HLA-DMB, HLA-DOB, HLA-DPB, HLA-DQB or HLA-DRB. In some embodiments, APCs present at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 T cell epitopes (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 T cell epitopes Table 1, Table 2, Table 3 and/or Table 4).

In some embodiments, the APCs are B cells, antigen presenting T-cells, dendritic cells, or artificial antigen-presenting cells (e.g., aK562 cells). Dendritic cells for use in the process may be prepared by taking PBMCs from a patient sample and adhering them to plastic. Generally the monocyte population sticks and all other cells can be washed off. The adherent population is then differentiated with IL-4 and GM-CSF to produce monocyte derived dendritic cells. These cells may be matured by the addition of IL-1β, IL-6, PGE-1 and TNF-α (which upregulates the important co-stimulatory molecules on the surface of the dendritic cell) and are then contacted with a recombinant adenovirus described herein.

In some embodiments, the APC is an artificial antigen-presenting cell, such as an aK562 cell. In some embodiments, the artificial antigen-presenting cells are engineered to express CD80, CD83, 41BB-L, and/or CD86. Exemplary artificial antigen-presenting cells, including aK562 cells, are described U.S. Pat. Pub. No. 2003/0147869, which is hereby incorporated by reference.

In certain aspects, provided herein are methods of generating APCs that present the two or more of the T cell epitopes described herein comprising contacting an APC with a nucleic acid vector and/or recombinant adenoviruses encoding T cell epitopes described herein and/or with a polyepitope produced by the nucleic acid vectors or recombinant adenoviruses described herein. In some embodiments, the APCs are irradiated.

### T Cells

In certain aspects, provided herein are T cells and populations of T cells (e.g., CD4 T cells and/or CD8 T cells) that express a TCR (e.g., an αβ TCR or a γδ TCR) that recognize a peptide described herein (e.g., an epitope listed in Table 1, Table 2, Table 3 and/or Table 4) presented on HLA. In some embodiments, the T cell is a CD8 T cell (a CTL) that expresses a TCR that recognizes a peptide described herein presented on a class I HLA. In some embodiments, the T cell is a CD4 T cell (a helper T cell) that recognizes a peptide described herein presented on a class II HLA. Most preferably, the present disclosure relates to the stimulation and expansion of polyfunctional T-cells, i.e., those T cells that are capable of inducing multiple immune effector functions, that provide a more effective immune response to an epitope (e.g., an epitope listed in Table 1, Table 2, Table 3 and/or Table 4) than do cells that produce, for example, only a single immune effector (e.g., a single biomarker such as a cytokine or CD107a). Less-polyfunctional, monofunctional, or even “exhausted” T cells may dominate immune responses during chronic infections or disease states (e.g., cancer), thus negatively impacting treatment or protection against virus-associated complications. The functional competence and activity of such T cells may be further assessed by determining the expression patterns (e.g., expression profiles by ICS assay) of transcription factors such as T-bet and Eomes and/or cytotoxic effector molecules such as perforin and Granzyme B. In some embodiments, the expression of each of T-bet, Eomes, perforin, and Granzyme B is determined for the T cells disclosed herein. Such expression levels may be determined and assessed as a relative measurement such as a ratio. In preferred embodiments, the expression profile of T-bet/Eomes and/or Granzyme B/perforin is determined. In preferred embodiments, the T cells disclosed herein (e.g., JCV-specific T cells) exhibit high expression of T-bet and low expression of Eomes (i.e., T-bet^{hi}/Eomes^{low}); similarly, the T cells disclosed herein may exhibit high expression of Granzyme B and low expression of perform (i.e., Granzyme^{hi}/Perforin^{low}). In some such embodiments, T cells (e.g., JCV-specific T cells) exhibiting a T-bet^{hi}/Eomes^{low} and/or a Granzyme^{hi}/Perforin^{low} expression profile are identified as functionally competent and active. Such T cells may be selected for expansion and/or use in an adoptive T cell immunotherapy. Most preferably, the T cells disclosed herein (e.g., JCV-specific T cells) are polyfunctional (i.e., produce 2 or more cytokines as described herein) and exhibit a T-bet^{hi}/Eomes^{low} and/or a Granzyme^{hi}/Perforin^{low} expression profile.

In some aspects, provided herein are methods of generating, activating and/or inducing proliferation of T cells (e.g., CTLs) that recognize one or more of the epitopes described herein. In some embodiments, a sample comprising CTLs (i.e., a PBMC sample) is incubated in culture with an APC provided herein (e.g., an APC that presents a peptide comprising a BKV and/or JCV epitope described herein on a class I HLA complex). In some embodiments, the sample containing T cells are incubated 2 or more times with APCs provided herein. In some embodiments, the T cells are incubated with the APCs in the presence of at least one cytokine. In some embodiments, the cytokine is IL-4, IL-7 and/or IL-15. Exemplary methods for inducing proliferation of T cells using APCs are provided, for example, in U.S. Pat. Pub. No. 2015/0017723, which is hereby incorporated by reference.

In some aspects, provided herein is a population of CTLs collectively comprising T cell receptors that recognize one or more T cell epitopes (e.g., one or more of the T cell epitopes listed in Table 1, Table 2, Table 3 and/or Table 4). In some embodiments, the CTLs recognize two or more T cell epitopes from Table 1, Table 2, Table 3 and/or Table 4. In some embodiments, the population of CTLs collectively comprise T cell receptors that recognize T cell epitopes from any combination of JCV, BKV, EBV, CMV, ADV and/or from other viruses. In some embodiments, the population of CTLs collectively comprise T cell receptors that recognize at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 T cell epitopes (e.g., at least 1, 2, 3, 4, 5, 6, or 7 T cell epitopes from Table 1 and/or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the epitopes listed in Table 1, Table 2, Table 3 and/or Table 4).

In some aspects, provided herein are methods of preventing or treating a polyomavirus infection (e.g., a JCV infection) or cancer (e.g., a polyomavirus associated cancer, such as a JVC associated cancer) in a subject comprising administering, to a subject, compositions (e.g., therapeutic compositions) comprising the nucleic acid vector described herein, peptides produced by the nucleic acid vector described herein, CTLs and/or APCs provided herein (e.g., comprising the nucleic acid vector described herein) and a pharmaceutically acceptable carrier. In some embodiments, the CTLs and/or APCs are not autologous to the subject (i.e., the CTLs and/or APCs are allogeneic to the subject). In some embodiments, the T cells and/or APCs are autologous to the subject. In some embodiments, the T cells and/or APCs are stored in a cell bank before they are administered to the subject.

### Pharmaceutical Compositions

In some aspects, provided herein is a composition (e.g., a pharmaceutical composition, such as a vaccine composition), containing a peptide (e.g., comprising an epitope from Table 1), nucleic acid, nucleic acid vector, recombinant adenovirus, antibody, CTL, or an APC described herein formulated together with a pharmaceutically acceptable carrier, as well as methods of treating cancer (e.g., a polyomavirus associated cancer, such as a JVC associated cancer) or a polyomavirus infection (e.g., a JCV, CMV, EBV, or ADV infection) using such pharmaceutical compositions. In some embodiments, the composition includes a combination of multiple (e.g., two or more) agents provided herein.

In some embodiments, the pharmaceutical composition further comprises an adjuvant. As used herein, the term “adjuvant” broadly refers to an agent that affects an immunological or physiological response in a patient or subject. For example, an adjuvant might increase the presence of an antigen over time or to an area of interest like a tumor, help absorb an antigen-presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent. Examples of adjuvants include, but are not limited to, an immune modulatory protein, Adjuvant 65, α-GalCer, aluminum phosphate, aluminum hydroxide, calcium phosphate, β-Glucan Peptide, CpG oligodeoxynucleotides, non-CpG oligodeoxynucleotides, GPI-0100, lipid A and modified versions thereof (e.g., monophosphorylated lipid A), lipopolysaccharide, Lipovant, Montanide, N-acetyl-muramyl-L-alanyl-D-isoglutamine, Pam3CSK4, quil A, a TLR9 agonist, ODN1a, a cationic antimicrobial peptide (CAMP) such as KLK, IC31, and trehalose dimycolate.

Methods of preparing these formulations or compositions include bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions of this invention suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents of the present invention, which may be used in a suitable hydrated form, and/or the pharmaceutical compositions of the present invention, are formulated into pharmaceutically-acceptable dosage forms by conventional methods known to those of skill in the art.

### Therapeutic Methods

JCV sequences and/or protein expression can be observed in several malignancies, and frequently reported in immunocompromised (e.g., immunodeficient, immuno-incompetent, and/or immunosuppressed) patients with and without PML. Accordingly, in certain aspects, provided herein are methods of treating and/or preventing cancer (e.g., a polyomavirus-associated cancer, such as a JCV-associated cancer) or a polyomavirus infection (e.g., a JCV infection). In some embodiments, the method comprises administering to the subject pharmaceutical composition comprising a CTL, APC, polypeptide and/or nucleic acid molecule described herein.

In some embodiments, the subject treated is immunocompromised. For example, in some embodiments, the subject has a T cell deficiency. In some embodiments, the subject has leukemia, lymphoma (e.g., Hodgkin's lymphoma) or multiple myeloma. In some embodiments, the subject has multiple sclerosis, psoriasis, and/or other autoimmune diseases. In some embodiments, the subject is infected with HIV and/or has AIDS. In some embodiments, the subject has undergone a tissue, organ and/or bone marrow transplant. In some embodiments, the subject is receiving immunosuppressive therapy such as steroids, cytostatics and antiproliferative agents, therapeutic antibodies, calcineurin inhibitors, anti-rejection drugs, and the like or combinations thereof. In some embodiments, the subject has undergone and/or is undergoing chemotherapy. In some embodiments, the subject has undergone and/or is undergoing radiation therapy.

In preferred embodiments, the subject is suffering from a JCV infection in the central nervous system (e.g. re-activation of a JCV infection or seeding of newly reactivated virus). In some such embodiments, the JCV infection is associated with destruction of oligodendrocytes and/or white matter demyelination. In further embodiments, the subject is suffering from JCV granule cell layer neuronopathy (JCV GCN), JCV encephalopathy (JCV CPN/JCVE), JCV meningitis (JCVM), and/or progressive multifocal leukoencephalopathy (PML), preferably from PML. In some such embodiments, the pathogen (e.g., JCV) is detectable in the cerebrospinal fluid of the subject.

In some embodiments, the subject has cancer. In some embodiments, the methods described herein may be used to treat any cancerous or pre-cancerous tumor. In some embodiments, the cancer expresses one or more of the polyomavirus epitopes provided herein (e.g., the BKV/JCV epitopes listed in Tables 1, 2, 3, and/or 4). In some embodiments, the cancer is a JVC-associated carcinoma. In some embodiments, the cancer includes a solid tumor. Preferably the cancer is a gastrointestinal malignancy, such as colon cancer, gastric cancer, gastrointestinal tumors, and the like. Most preferably, the cancer is a CNS malignancy, such as gliomas and all subtypes thereof (e.g., ependymomas, astrocytomas, brainstem gliomas, oligodendrogliomas, optic nerve gliomas, mixed gliomas, and the like), medulloblastomas, primitive neuroectodernal tumors, and neuroblastomas. For the purpose of exemplification without limitation, cancers that may be treated by methods and compositions provided herein includecancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous iadenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; glioblastoma multiforme; pineoblastoma, gliosarcoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject is also administered an anti-viral drug that inhibits polyomavirus replication. For example, in some embodiments, the subject is administered ganciclovir, valganciclovir, foscarnet, cidofovir, acyclovir, formivirsen, maribavir, BAY 38-4766 or GW275175X.

In some embodiments, the subject is also administered an immune checkpoint inhibitor. Immune Checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, atezolizumab, avelumab, camrelizumab, cemiplimab, cetrelimab, durvalumab (MEDI-4736), genolimzumab, ipilimumab, nivolumab, pembrolizumab, pidilizumab, sintilimab, spartalizumab, tislelizumab, Toripalimab, AMP-224, AMP-514, AK-104, ASP-8374, AUR-012, BCD-135, BGB-A333, BMS-936559, CBT-502, MCLA-145, KN-046, MGD-019, MK-4830, MSB-0020718C, RG-7446, SL-279252, STI-A1010, STI-A1110, TSR-042, XmAb20717, and XmAb23104.

In some embodiments, a composition provided herein is administered prophylactically to prevent cancer and/or a polyomavirus infection (e.g., JCV infection). In some embodiments the composition may be administered prior to or after the detection of cancer cells or polyoma virus-infected cells in a subject. Accordingly, in some such embodiments, a composition provided herein is administered prior to or after the administration of an immunosuppressive therapy (e.g., steroids, cytostatics and antiproliferative agents, therapeutic antibodies, calcineurin inhibitors, anti-rejection drugs, and the like or combinations thereof). In some such embodiments, the composition is administered prior to or after chemotherapy. Likewise, in some embodiments, the composition is administered prior to or after radiation therapy. In some embodiments, after administration of a composition comprising peptides, nucleic acids, CTLs, and/or APCs described herein, a proinflammatory response is induced. The proinflammatory immune response comprises production of proinflammatory cytokines and/or chemokines, for example, interferon gamma (IFN-γ) and/or interleukin 2 (IL-2).

Conjunctive therapy includes sequential, simultaneous and separate, and/or co-administration of the active compounds in such a way that the therapeutic effects of the first agent administered have not entirely disappeared when the subsequent treatment is administered. In some embodiments, the second agent may be co-formulated with the first agent or be formulated in a separate pharmaceutical composition.

In some aspects, provided herein is a method of identifying a subject suitable for a therapy provided herein (e.g., methods of treating a polyoma virus infection, such as JCV infection, and/or cancer in a subject comprising administering to the subject a pharmaceutical composition provided herein). In some embodiments, the method comprises isolating a sample from the subject (e.g., a blood sample, a tissue sample, a tumor sample) and detecting the presence of an epitope listed in Tables 1, 2 or 3 in the sample. In some embodiments the epitope is detected using an ELISA assay, a western blot assay, a FACS assay, a fluorescent microscopy assay, an Edman degradation assay and/or a mass spectrometry assay (e.g., protein sequencing). In some such embodiments, the presence of a JCV epitope, for example, is detected by detecting a nucleic acid encoding the JCV epitope. In some embodiments, the nucleic acid encoding the JCV epitope is detected using a nucleic acid probe, a nucleic acid amplification assay and/or a sequencing assay. Notably, the JC viral genome is composed of two conserved coding regions separated by a highly variable non-coding control region (NCCR) harboring both sequences required for replication (ORI, the origin of viral replication) and for transcription (several promoters and cis-regulating elements); a highly conserved region which includes ORI is followed by sections a, b, c, d, e and f JC virus found in the CNS of PML patients is often found to have rearranged NCCRs (e.g., absence of b and d sections and duplication of the a-c-e sequence). Differences in NCCR sequence may contribute to the fitness of the virus in the CNS and thus to the development of PML. Accordingly, provided herein are methods of identifying a subject suitable for a therapy provided herein, comprising isolating a sample from the subject (e.g., a blood sample, a urine sample, a tissue sample, a cerebrospinal fluid sample, a tumor sample) and detecting the presence of PML-associated JCV sequence rearrangements (e.g. using nucleic acid amplification techniques such as nested PCR and the like). Such sequences and methods of detection are known in the art, such as in L'Honner et al., PLoS ONE, 13(6), 2018 which is incorporated by reference in its entirety.

In some embodiments, the method comprises HLA typing of the subject. In some embodiments, the subject is identified as suitable for treatment with a method provided herein if the subject expresses an HLA to which an epitope provided herein is restricted. In some embodiments, the methods provided herein further comprise treating the identified subject using a therapeutic method provided herein (e.g., by administering to the subject a pharmaceutical composition provided herein). In some embodiments, the subject is administered a composition comprising CTLs described herein, wherein the CTLs comprise TCRs that recognize an epitope provided herein that is HLA restricted to an HLA expressed by the subject. In some embodiments, the subject is administered a composition comprising a polypeptide comprising an epitope provided herein that is HLA restricted to an HLA expressed by the subject. In some embodiments, the subject is administered a composition comprising an APC presenting a polypeptide comprising an epitope provided herein that is HLA restricted to an HLA expressed by the subject. In some embodiments, the subject is administered a composition comprising an nucleic acid encoding a polypeptide comprising an epitope provided herein that is HLA restricted to an HLA expressed by the subject.

### EXAMPLES

### Example 1: CD8⁺ and CD4⁺ T Cell Responses Directed Towards LTA, VP1 and STA JCV Antigens

PBMCs from 17 healthy volunteers were incubated with JVC overlapping peptide pools (OPPs) and cultured for 14 days in the presence of IL-2. Peptide matrices for each of large T antigen (LTA), small T antigen (STA), and viral protein 1 (VP1), as well as the composition of the peptide pools for each matrix, were arranged as follows.

### Large T Antigen Matrix

| LTA | LTA | LTA | LTA | LTA | LTA | LTA | LTA | LTA | LTA | LTA |
|---|---|---|---|---|---|---|---|---|---|---|
| Pools | 15 | 16 | 17 | 18 | 19 | 20 | 21 | 22 | 23 | 24 |
| LTA 1 | P1 | P2 | P3 | P4 | P5 | P6 | P7 | P8 | P9 | P10 |
| LTA 2 | P11 | P12 | P13 | P14 | P15 | P16 | P17 | P18 | P19 | P20 |
| LTA 3 | P21 | P22 | P23 | P24 | P25 | P26 | P27 | P28 | P29 | P30 |
| LTA 4 | P31 | P32 | P33 | P34 | P35 | P36 | P37 | P38 | P39 | P40 |
| LTA 5 | P41 | P42 | P43 | P44 | P45 | P46 | P47 | P48 | P49 | P50 |
| LTA 6 | P51 | P52 | P53 | P54 | P55 | P56 | P57 | P58 | P59 | P60 |
| LTA 7 | P61 | P62 | P63 | P64 | P65 | P66 | P67 | P68 | P69 | P70 |
| LTA 8 | P71 | P72 | P73 | P74 | P75 | P76 | P77 | P78 | P79 | P80 |
| LTA 9 | P81 | P82 | P83 | P84 | P85 | P86 | P87 | P88 | P89 | P90 |
| LTA 10 | P91 | P92 | P93 | P94 | P95 | P96 | P97 | P98 | P99 | P100 |
| LTA 11 | P101 | P102 | P103 | P104 | P105 | P106 | P107 | P108 | P109 | P110 |
| LTA 12 | P111 | P112 | P113 | P114 | P115 | P116 | P117 | P118 | P119 | P120 |
| LTA 13 | P121 | P122 | P123 | P124 | P125 | P126 | P127 | P128 | P129 | P130 |
| LTA 14 | P131 | P132 | P133 | P134 | P135 | P136 | P137 | | | |

### Small T Antigen

| VP1 | VP | VP | VP | VP | VP | VP | VP | VP |
|---|---|---|---|---|---|---|---|---|
| pools | 10 | 11 | 12 | 13 | 14 | 15 | 16 | 17 |
| VP 1 | V1 | V2 | V3 | V4 | V5 | V6 | V7 | V8 |
| VP 2 | V9 | V10 | V11 | V12 | V13 | V14 | V15 | V16 |
| VP 3 | V17 | V18 | V19 | V20 | V21 | V22 | V23 | V24 |
| VP 4 | V25 | V26 | V27 | V28 | V29 | V30 | V31 | V32 |
| VP 5 | V33 | V34 | V35 | V36 | V37 | V38 | V39 | V40 |
| VP 6 | V41 | V42 | V43 | V44 | V45 | V46 | V47 | V48 |
| VP 7 | V49 | V50 | V51 | V52 | V53 | V54 | V55 | V56 |
| VP 8 | V57 | V58 | V59 | V60 | V61 | V62 | V63 | V64 |
| VP 9 | V65 | V66 | V67 | V68 | V69 | V70 | V71 | |

### Viral Protein 1

| STA | | | | | | |
|---|---|---|---|---|---|---|
| Pools | STA 7 | STA 8 | STA 9 | STA 10 | STA 11 | STA 12 |
| STA 1 | S1 | S2 | S3 | S4 | S5 | S6 |
| STA 2 | S7 | S8 | S9 | S10 | S11 | S12 |
| STA 3 | S13 | S14 | S15 | S16 | S17 | S18 |
| STA 4 | S19 | S20 | S21 | S22 | S23 | S24 |
| STA 5 | S25 | S26 | S27 | S28 | S29 | S30 |
| STA 6 | S31 | S32 | S33 | | | |

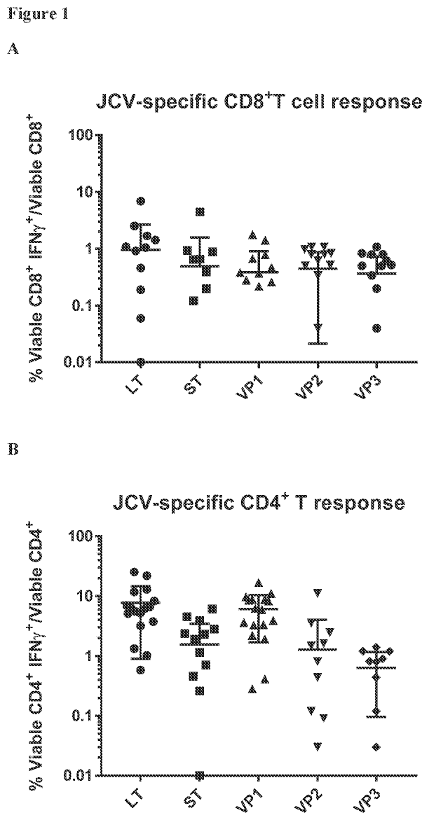

On day 14, these T cell cultures were assessed for JVC-specificity using an intracellular cytokine (ICS) assay. Notably, in vitro culture of T cells with JVC peptides for 14 days resulted in expansion of virus-specific T cells. These initial analyses clearly showed that T cell responses were directed towards LTA, VP1 and STA (see FIG. 1).

### Example 2: JCV Epitope HLA Restriction

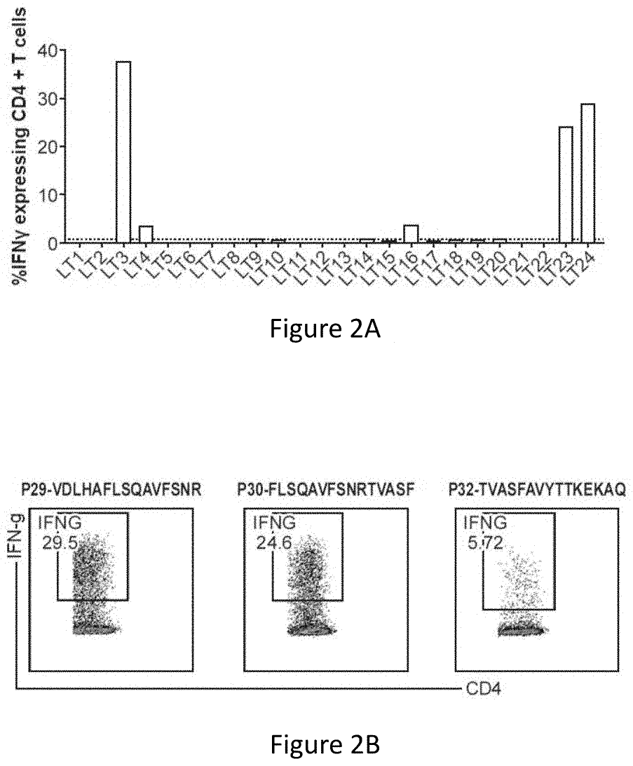

In order to precisely map the HLA class I and class II-restricted T cell responses, individual overlapping peptides (15 aa long overlapping by 10 aa) were sourced for LTA, STA and VP1 proteins for T cell epitope mapping. A two-dimensional peptide matrix was used to distribute all individual peptides into small overlapping peptide pools. For example, in the case of Large T Antigen, the matrix is arranged such that each peptide of the pools (LTA1 to LTA24) occurs once on the ordinate. The T cell response for each pool was measured by intracellular cytokine-staining (ICS) IFN-γ assay (see FIG. 2A) and the data overlayed on a two dimensional matrix as follows.

### Peptide Pool LTA1-LTA24 T Cell Response Matrix

| | LTA15 | [symbol] | LTA17 | LTA18 | LTA19 | LTA20 | LTA21 | LTA22 | [symbol] | [symbol] |
|---|---|---|---|---|---|---|---|---|---|---|
| LTA1 | P1 | P2 | P3 | P4 | P5 | P6 | P7 | P8 | P9 | P10 |
| LTA2 | P11 | P12 | P13 | P14 | P15 | P16 | P17 | P18 | P19 | P20 |
| [symbol] | P21 | P22 | P23 | P24 | P25 | P26 | P27 | P28 | [symbol] | [symbol] |
| [symbol] | P31 | [symbol] | P33 | P34 | P35 | P36 | P37 | P38 | P39 | P40 |
| LTA5 | P41 | P42 | P43 | P44 | P45 | P46 | P47 | P48 | P49 | P50 |
| LTA6 | P51 | P52 | P53 | P54 | P55 | P56 | P57 | P58 | P59 | P60 |
| LTA7 | P61 | P62 | P63 | P64 | P65 | P66 | P67 | P68 | P69 | P70 |
| LTA8 | P71 | P72 | P73 | P74 | P75 | P76 | P77 | P78 | P79 | P80 |
| LTA9 | P81 | P82 | P83 | P84 | P85 | P86 | P87 | P88 | P89 | P90 |
| LTA10 | P91 | P92 | P93 | P94 | P95 | P96 | P97 | P98 | P99 | P100 |
| LTA11 | P101 | P102 | P103 | P104 | P105 | P106 | P107 | P108 | P109 | P110 |
| LTA12 | P111 | P112 | P113 | P114 | P115 | P116 | P117 | P118 | P119 | P120 |
| LTA13 | P121 | P122 | P123 | P124 | P125 | P126 | P127 | P128 | P129 | P130 |
| LTA14 | P131 | P132 | P133 | P134 | P135 | P136 | | | | |

Thus, the common individual peptides among pools that elicited a T cell response were identified, i.e., peptide 32 (P32) at the intersection of row LTA4 and column LTA16 and peptides P29 and P30 at the intersection of row LTA3 with columns LTA23 and LTA24. Fluorescence-activated cell sorting (FACS) confirms that individual peptides P29, P30, and P32 elicit a JCV-specific T cell response (see FIG. 2B)

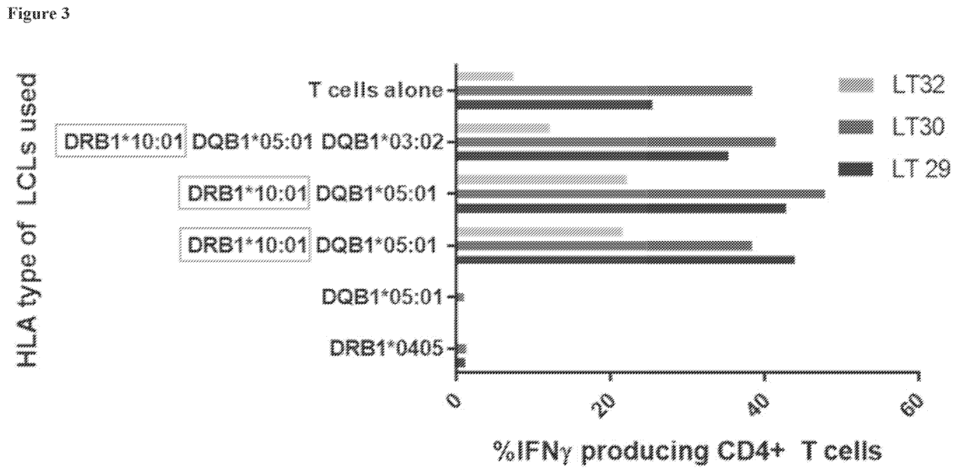

These individual peptides were further assessed for T cell expansion and ICS analysis to identify potential JCV antigens. The resultant peptides provide a high percentage of HLA allele coverage for JCV (see FIG. 3 and Table 5).

### Table 5: HLA Restriction Mapping and Allele Coverage

| | | | HLA | |
|---|---|---|---|---|
| | JCV | | Allele | SEQ ID |
| Peptide sequence | Antigen | HLA Restriction | coverage | NO.: |
| IDQFMVVFEDVKGTG | LTA | DRB1*14:04/ | 7.45% | 1 |
| | | DQB1*05:03 | | |
| VDLHAFLSQAVFSNR | LTA | DRB1*10:01 | 2.59% | 2 |
| FLSQAVFSNRTVASF | LTA | | | 3 |
| TVASFAVYTTKEKAQ | LTA | | | 4 |
| ERLNFELGVGIDQFM | LTA | DRB1*15:01 | 18.41% | 5 |
| TCGNILMWEAVTLKT | VP1 | | | 6 |
| RYWLFKGPIDSGKTT | LTA | | | 7 |
| MTREEMLVERFNFLL | LTA | DRB1*04:01 & | 27.90% | 8 |
| EQYMAGVAWIHCLLP | LTA | DRB1*03:01 | | 9 |
| RKAYLKKCKELHPDK | STA | DRB1*13:01 | 8.77% | 10 |
| GGHNILFFLTPHRHR | LTA | DRB1*16:O1 | 4.61 | 11 |
| RSGSQQWRGLSRYFK | VP1 | DRB1*11:01 | 10.54% | 12 |
| DPDMMRYVDKYGQLQ | VP1 | DRB1*04:02 | 1.85% | 13 |
| MDKVLNREESMELMD | LTA | DRB1*01:01 | 11.53% | 14 |
| SITEVECFLTPEMGD | VP1 | DRB1*01:01 & | 13.62% | 15 |
| | | DQB1*05:01 | | |
| SKNQKSICQQAVDTV | LTA | DRB1*04:01 & | 11.2% | 16 |
| SICQQAVDTVAAKQR | LTA | DQB1*02:02 | 12.44% | 17 |
| RNRKFLRSSPLVWID | LTA | DRB1*15:01 | 18.41% | 18 |
| LRSSPLVWIDCYCFD | LTA | | | 19 |
| KMKRMNFLYKKMEQG | LTA | | | 20 |
| NFLYKKMEQGVKVAH | LTA | | | 21 |

### Example 3: JCV-Specific T Cell Expansion and Characterization

JCV-specific T cells were expanded in vitro following stimulation with pooled JCV epitopes. Specifically, PBMC from healthy volunteers were stimulated with synthetic JCV peptides (Table 1) for 1 hour and then cultured for 12-14 days in the presence of different cytokine combinations, including IL-2 (10 ng/ml), IL7 (10 ng/ml), IL12 (10 ng/ml) and/or IL15 (10 ng/ml). The JCV specificity of the expanded T cells was assessed using standard intracellular cytokine assays (Table 6).

### Table 6: CD4⁺ T Cell Responses to JCV Peptides

| | | %IFNγ⁺CD4⁺ | %IFNγ⁺CD4⁺ | | |
|---|---|---|---|---|---|
| | JCV | with 1 μg/ml | with 0.1 μg/ml | BKV cross- | SEQ ID |
| Peptide sequence | Antigen | peptide | peptide | reactivity | NO.: |
| IDQFMVVFEDVKGTG | LTA | 24.8 | 18.5 | Yes | 1 |
| VDLHAFLSQAVFSNR | LTA | 25.49 | 20.26 | Yes | 2 |
| FLSQAVFSNRTVASF | LTA | 38.39 | 34.1 | Yes | 3 |
| TVASFAVYTTKEKAQ | LTA | 7.34 | 6.4 | Yes | 4 |
| ERLNFELGVGIDQFM | LTA | 6.32 | 2.2 | Yes | 5 |
| TCGNILMWEAVTLKT | VP1 | 4.83 | 2.65 | Yes | 6 |
| RYWLFKGPIDSGKTT | LTA | 4.05 | 2.35 | Yes | 7 |
| MTREEMLVERFNFLL | LTA | 3.8 | 2 | Yes | 8 |
| EQYMAGVAWIHCLLP | LTA | 2.9 | 2.5 | Yes | 9 |
| RKAYLKKCKELHPDK | STA | 10.4 | 9.2 | Yes | 10 |
| GGHNILFFLTPHRHR | LTA | 6.15 | 1.28 | Yes | 11 |
| RSGSQQWRGLSRYFK | VP1 | 8.4 | 7.8 | Yes | 12 |
| DPDMMRYVDKYGQLQ | LTA | 4.5 | 2.1 | Yes | 13 |
| MDKVLNREESMELMD | LTA | 5.26 | 1.65 | Yes | 14 |
| SITEVECFLTPEMGD | VP1 | 2.71 | 2.8 | Yes | 15 |
| SKNQKSICQQAVDTV | LTA | 2.31 | 0.41 | Yes | 16 |
| SICQQAVDTVAAKQR | LTA | 4.38 | 0.24 | Yes | 17 |
| RNRKFLRSSPEVWID | LTA | 3.03 | 0.6 | N/A | 18 |
| LRSSPLVWIDCYCFD | LTA | 1.81 | 0.4 | N/A | 19 |
| KMKRMNFLYKKMEQG | LTA | 1.4 | 0.4 | Yes | 20 |
| NFLYKKMEQGVKVAH | LTA | 1.2 | 0.2 | N/A | 21 |

### Example 4: T Cell Cross-Reactivity Between JCV and BKV Epitopes

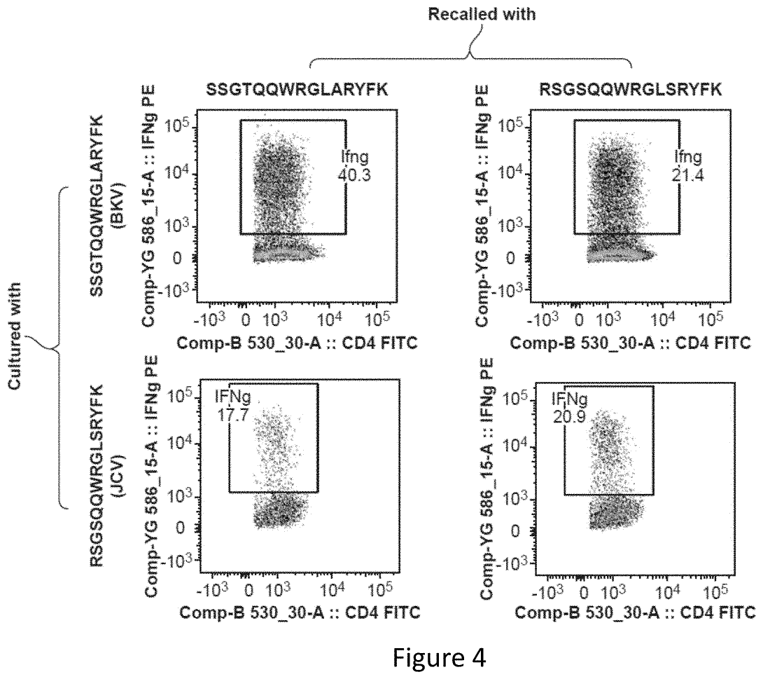

Peptide-specific T cells were expanded in vitro following stimulation with JCV epitope or BKV epitope and then re-stimulated with the corresponding homologous peptide epitope (see Table 2) so as to observe any cross-reactive response between JCV and BKV epitopes. Specifically, PBMC from healthy volunteers were cultured with synthetic JCV peptide epitope RSGSQQWRGLSRYFK (SEQ ID NO: 12) or with synthetic BKV peptide epitope SSGTQQWRGLARYFK (SEQ ID NO: 33). Following initial expansion, each sample was re-stimulated (re-called) with either JCV epitope RSGSQQWRGLSRYFK (SEQ ID NO: 12) or BKV epitope SSGTQQWRGLARYFK (SEQ ID NO: 33) (wherein samples re-called with the same epitope act as internal controls). The reactivity of the expanded T cells was assessed using standard intracellular cytokine assays (see FIG. 4). T cells expanded with either of the homolgous epitopes recognize both the BKV and JCV peptide sequence.

### Example 5: Profiling Functional and Phenotypic Characteristics of JCV-Specific T Cells in Healthy Individuals and Transplant Recipients

In recent years, the T-box transcription factors (T-bet) and Eomesodermin (Eomes) have been shown to play important roles in determining the fate of CD8⁺ T cells during infection. High levels of T-bet are associated with the cytotoxic T cell differentiation and upregulation of perforin and Granzyme B in antigen specific cells. A high level of Eomes is associated with the long-term memory formation. It has been seen in various studies that their cooperative expression is critical for infection control. In mouse studies, it has also been shown that the deletion of either of the transcription factors results in failure to control infection. Hence it is critical to study the expression of these transcription factors which could help in understanding the phenotypic characterization of T cells and T cell differentiation during both acute and chronic viral infections. The expression patterns of T-bet and Eomes in JCV-specific T cells is not yet understood, and the analysis of these transcription factors on such T cells may enable a deeper understanding on the differentiation of JCV-specific T cells. A detailed study on the functional characteristics of T cells could also lead to development of effective immunotherapy for JCV associated diseases. An initial set of experiments studies the transcriptional factors on the T cells which regulate their differentiation. The expression of T-bet, Eomes, perforin and granzyme B are assayed on JCV-specific T cells and CMV specific T cells using ICS. Initial analysis shows a medium to low level of T bet expression in JCV-specific T cells while high levels of T-bet are seen with CMV specific T cells. Very low expression of Eomes is found with JCV-specific T cells in comparison to the CMV specific T cells. Likewise, low levels of perforin and granzyme B are also seen with JCV-specific T cells. This suggests that, relative to CMV-specific T cells, JCV-specific T cells are functionally low in effector function. Hence, driving the effector function of JCV-specific CTLs is the focus of studies contributing to the development of an effective adoptive T cell immunotherapy.

### Example 6: Feasibility of T Cell Expansion with Proposed Peptide Pool

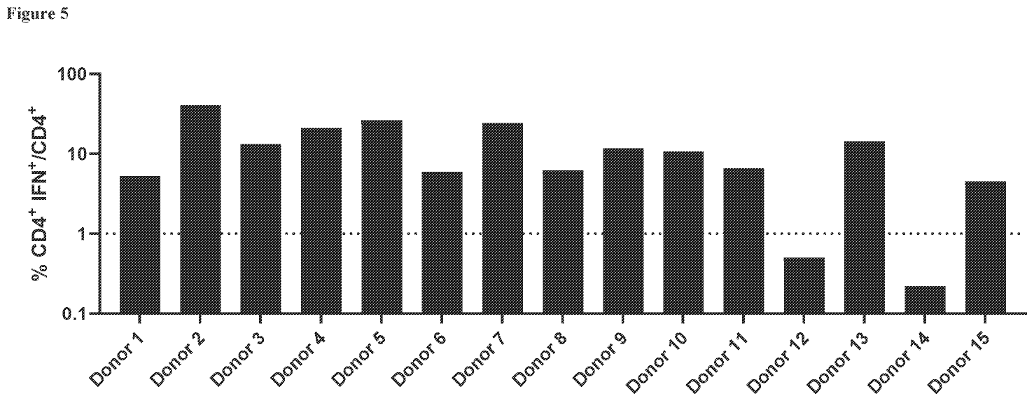

PBMCs from 15 healthy donors were selected randomly irrespective of their HLA type and JCV-specific T cells were expanded upon stimulation with a pool of peptides comprising the peptides disclosed herein (i.e., peptides comprising the amino acid sequences set forth in SEQ ID NOs. 1-21). The T cells were expanded for 17 days and assessed for JCV response using intracellular cytokine staining assay. T cells from thirteen out of fifteen donors had JCV-specific T cell response which is evidenced by the production of IFN-γ upon re-stimulation with the peptide pool (see FIG. 5).

### Example 7: Functional Characterization of JCV-Specific T Cell Product

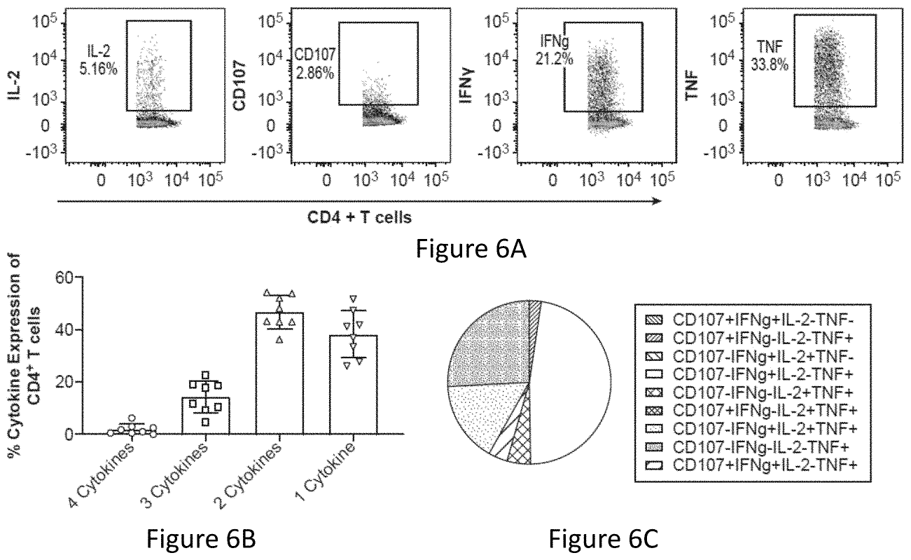

To determine the polyfunctionality of JCV-specific T cells expanded using the peptide pool, T cells were analysed for their expression of IL-2, TNF, IFN-γ and CD107 by intracellular staining (see FIG. 6a). JCV-specific T cells showed a higher expression of TNF along with other cytokines.

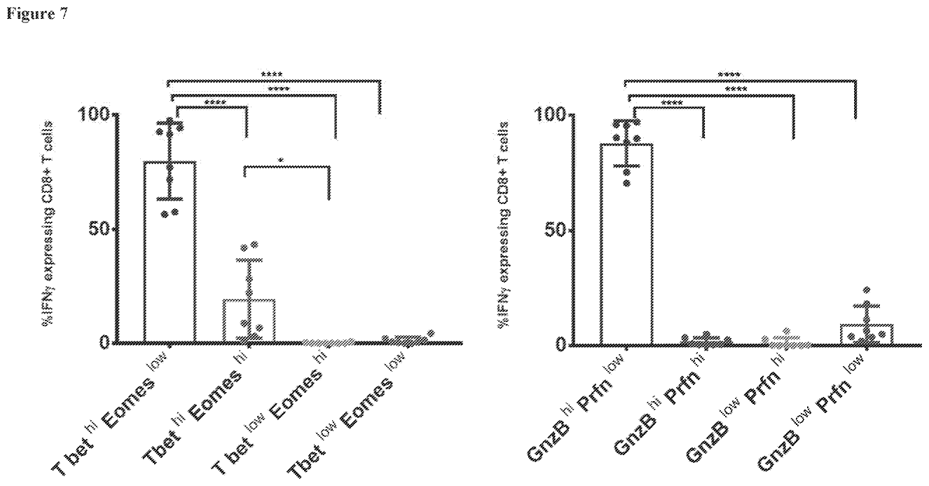

Boolean analysis for the expression pattern of different cytokine combinations revealed JCV-specific T cell products were polyfunctional and produced 2 or more cytokines (see FIG. 6, b and c). To further characterize JCV-specific T cell product, the expression of transcription factors (T-bet and Eomes) and effector molecules (perforin and granzyme B). A large proportion of cells had a T-bet^{hi}/Eomes^{low} and Granzyme^{hi}/Perforin^{low} profile which revealed that JCV-specific T cells are functionally active and capable of showing cytotoxic effect against JCV-infected cells. Such T cells can be expanded in vitro and used for treating JCV-associated diseases (see FIG. 7).

1. A peptide comprising one or more of the epitopes listed in Tables 1-4.

2. (canceled)

3. The peptide of claim 1, wherein the one or more epitopes comprises JCV epitopes set forth in SEQ ID NOs: 1-21.

4-5. (canceled)

6. The peptide of claim 1, wherein the peptide comprises a plurality of epitopes listed in Tables 1-4.

8. The peptide of claim 6, wherein the plurality of epitopes comprise a plurality of JCV epitopes set forth in SEQ ID NOs: 1-21.

9-25. (canceled)

26. A pharmaceutical composition comprising the peptide of claim 1 and a pharmaceutically acceptable carrier.

27. (canceled)

28. A vaccine composition comprising the peptide of claim 1 and a pharmaceutically acceptable carrier.

29. (canceled)

30. A method of treating or preventing a polyomavirus infection in a subject, comprising administering to the subject the pharmaceutical composition of claim 26.

31. The method of claim 30, wherein the polyomavirus infection is a JC virus (JCV) infection.

32. (canceled)

33. A method of treating or preventing a polyomavirus-associated cancer in a subject, comprising administering to the subject the pharmaceutical composition of claim 26.

34. The method of claim 33, wherein the polyomavirus-associated cancer is a JCV-associated cancer.

35-37. (canceled)

38. A pool of immunogenic peptides comprising HLA class I and/or class II-restricted JCV peptide epitopes capable of inducing proliferation of peptide-specific T cells, wherein the peptide pool comprises at least two of the epitope amino acid sequences set forth in SEQ ID NOs. 1-21, or combinations thereof.

39. (canceled)

40. The pool of immunogenic peptides of claim 38, wherein the peptide pool comprises at least ten of the JCV peptide epitope amino acid sequences set forth in SEQ ID NOs: 1-21.

41-46. (canceled)

47. A method of expanding JC virus-specific T cells for adoptive immunotherapy, comprising:
(i) contacting one or more cells isolated from a subject with the pool of immunogenic peptides of claim 38; and
(ii) culturing the one or more cells under conditions such that JC virus-specific T-cells are expanded from said one or more cells.

48. The method of claim 47, wherein the peptide or pool of immunogenic peptides consists essentially of at least ten of the JCV peptide epitope amino acid sequences set forth in SEQ ID NOs. 1-21.

49. The method of claim 47, wherein the one or more cells isolated from the subject comprises peripheral blood mononuclear cells (PBMCs) from a healthy donor or an immunocompromised donor.

50-55. (canceled)

56. A method of treating or preventing JCV infection in a subject, comprising administering to the subject the JC virus-specific T cells of claim 47.

57. A population of JCV-specific CTLs prepared by the method of claim 47 wherein the CTLs are collectively specific to at least two of the epitope amino acid sequences set forth in SEQ ID NOs. 1-21, or combinations thereof.

58-62. (canceled)

63. The method of claim 56, wherein the JCV infection is drug-resistant.

64-65. (canceled)

66. A method of treating or preventing a polyomavirus-associated cancer in a subject, comprising administering the expanded JC virus-specific T lymphocytes of claim 47.

67. (canceled)

68. A method of detecting a JC virus infection in a subject, the method comprising detecting the presence of JCV-specific T lymphocytes by contacting T lymphocytes isolated from the subject with the peptide of claim 1.

69. The method of claim 68, further comprising detecting JCV-specific DNA in a sample of cerebrospinal fluid isolated from the subject.

70. (canceled)

71. The method of claim 68, further comprising administering to the subject an adoptive immunotherapy composition comprising JC virus-specific T cells which target any one of the epitope amino acid sequences set forth in SEQ ID NOs: 1-21, or combinations thereof.

72-74. (canceled)

75. A method of treating or preventing a cancer or a polyomavirus infection in a subject, the method comprising administering to the subject a pharmaceutical composition comprising cytotoxic T cells (CTLs) comprising T cell receptors (TCRs) that recognize one or more epitopes listed in Tables 1-4.

76-108. (canceled)

109. A method of inducing proliferation of polyomavirus-specific cytotoxic T cells (CTLs), comprising contacting CTLs with antigen-presenting cells (APCs) that present one or more polyomavirus peptides comprising any one of the epitopes listed in Tables 1-4.

110-121. (canceled)

122. The method of claim 109, wherein the CTLs are from a sample of peripheral blood mononuclear cells (PBMCs).

123-124. (canceled)

125. A method of treating or preventing a cancer or a polyomavirus infection in a subject, the method comprising administering to the subject a vaccine composition comprising one or more epitopes listed in Tables 1-4.

126-153. (canceled)

154. A population of cytotoxic T cells (CTLs) that collectively comprise TCRs that recognize at least two of the epitopes listed in Tables 1-4.

155. The population of CTLs of claim 154, wherein the CTLs collectively comprise TCRs that recognize at least ten of the epitopes set forth in SEQ ID NOs: 1-21.


SEQUENCE LISTING (122 sequences)

SEQ ID NO: 1 - protein, 15 aa, Betapolyomavirus secuhominis
IDQFMVVFEDVKGTG

SEQ ID NO: 2 - protein, 15 aa, Betapolyomavirus secuhominis
VDLHAFLSQAVFSNR

SEQ ID NO: 3 - protein, 15 aa, Betapolyomavirus secuhominis
FLSQAVFSNRTVASF

SEQ ID NO: 4 - protein, 15 aa, Betapolyomavirus secuhominis
TVASFAVYTTKEKAQ

SEQ ID NO: 5 - protein, 15 aa, Betapolyomavirus secuhominis
ERLNFELGVGIDQFM

SEQ ID NO: 6 - protein, 15 aa, Betapolyomavirus secuhominis
TCGNILMWEAVTLKT

SEQ ID NO: 7 - protein, 15 aa, Betapolyomavirus secuhominis
RYWLFKGPIDSGKTT

SEQ ID NO: 8 - protein, 15 aa, Betapolyomavirus secuhominis
MTREEMLVERFNFLL

SEQ ID NO: 9 - protein, 15 aa, Betapolyomavirus secuhominis
EQYMAGVAWIHCLLP

SEQ ID NO: 10 - protein, 15 aa, Betapolyomavirus secuhominis
RKAYLKKCKELHPDK

SEQ ID NO: 11 - protein, 15 aa, Betapolyomavirus secuhominis
GGHNILFFLTPHRHR

SEQ ID NO: 12 - protein, 15 aa, Betapolyomavirus secuhominis
RSGSQQWRGLSRYFK

SEQ ID NO: 13 - protein, 15 aa, Betapolyomavirus secuhominis
DPDMMRYVDKYGQLQ

SEQ ID NO: 14 - protein, 15 aa, Betapolyomavirus secuhominis
MDKVLNREESMELMD

SEQ ID NO: 15 - protein, 15 aa, Betapolyomavirus secuhominis
SITEVECFLTPEMGD

SEQ ID NO: 16 - protein, 15 aa, Betapolyomavirus secuhominis
SKNQKSICQQAVDTV

SEQ ID NO: 17 - protein, 15 aa, Betapolyomavirus secuhominis
SICQQAVDTVAAKQR

SEQ ID NO: 18 - protein, 15 aa, Betapolyomavirus secuhominis
RNRKFLRSSPLVWID

SEQ ID NO: 19 - protein, 15 aa, Betapolyomavirus secuhominis
LRSSPLVWIDCYCFD

SEQ ID NO: 20 - protein, 15 aa, Betapolyomavirus secuhominis
KMKRMNFLYKKMEQG

SEQ ID NO: 21 - protein, 15 aa, Betapolyomavirus secuhominis
NFLYKKMEQGVKVAH

SEQ ID NO: 22 - protein, 15 aa, Betapolyomavirus hominis
IDQYMVVFEDVKGTG

SEQ ID NO: 23 - protein, 15 aa, Betapolyomavirus hominis
SDLHQFLSQAVFSNR

SEQ ID NO: 24 - protein, 15 aa, Betapolyomavirus hominis
FLSQAVFSNRTLACF

SEQ ID NO: 25 - protein, 15 aa, Betapolyomavirus hominis
TLACFAVYTTKEKAQ

SEQ ID NO: 26 - protein, 15 aa, Betapolyomavirus hominis
ERLTFELGVAIDQYM

SEQ ID NO: 27 - protein, 15 aa, Betapolyomavirus hominis
TCGNLLMWEAVTVKT

SEQ ID NO: 28 - protein, 15 aa, Betapolyomavirus hominis
RYWLFKGPIDSGKTT

SEQ ID NO: 29 - protein, 15 aa, Betapolyomavirus hominis
MTREEMLTERFNHIL

SEQ ID NO: 30 - protein, 15 aa, Betapolyomavirus hominis
EQYMAGVAWLHCLLP

SEQ ID NO: 31 - protein, 15 aa, Betapolyomavirus hominis
RKAYLRKCKEFHPDK

SEQ ID NO: 32 - protein, 15 aa, Betapolyomavirus hominis
AGHNIIFFLTPHRHR

SEQ ID NO: 33 - protein, 15 aa, Betapolyomavirus hominis
SSGTQQWRGLARYFK

SEQ ID NO: 34 - protein, 15 aa, Betapolyomavirus hominis
DPDMIRYIDRQGQLQ

SEQ ID NO: 35 - protein, 15 aa, Betapolyomavirus hominis
MDKVLNREESMELMD

SEQ ID NO: 36 - protein, 15 aa, Betapolyomavirus hominis
AITEVECFLNPEMGD

SEQ ID NO: 37 - protein, 15 aa, Betapolyomavirus hominis
SKNQKSICQQAVDTV

SEQ ID NO: 38 - protein, 15 aa, Betapolyomavirus hominis
SICQQAVDTVLAKKR

SEQ ID NO: 39 - protein, 15 aa, Betapolyomavirus hominis
LNRKFLRKEPLVWID

SEQ ID NO: 40 - protein, 15 aa, Betapolyomavirus hominis
LRKEPLVWIDCYCID

SEQ ID NO: 41 - protein, 15 aa, Betapolyomavirus hominis
KMKRMNTLYKKMEQD

SEQ ID NO: 42 - protein, 15 aa, Betapolyomavirus hominis
NTLYKKMEQDVKVAH

SEQ ID NO: 43 - protein, 10 aa, Betapolyomavirus secuhominis
  MOD_RES (3)..(3): Q or R
  MOD_RES (9)..(9): K or R
KSXHSTPPXK

SEQ ID NO: 44 - protein, 9 aa, Betapolyomavirus secuhominis
AVDTVAAKQ

SEQ ID NO: 45 - protein, 9 aa, Betapolyomavirus secuhominis
CYCFDCFRQ

SEQ ID NO: 46 - protein, 9 aa, Betapolyomavirus secuhominis
IPVMRKAYL

SEQ ID NO: 47 - protein, 9 aa, Betapolyomavirus secuhominis
FPPNSDTLY

SEQ ID NO: 48 - protein, 9 aa, Betapolyomavirus secuhominis
FLYCKEWPN

SEQ ID NO: 49 - protein, 9 aa, Betapolyomavirus secuhominis
  MOD_RES (5)..(5): W or R
SPLVXIDCY

SEQ ID NO: 50 - protein, 10 aa, Betapolyomavirus secuhominis
VHCPCLMCML

SEQ ID NO: 51 - protein, 12 aa, Betapolyomavirus secuhominis
NREESMELMDLL

SEQ ID NO: 52 - protein, 9 aa, Betapolyomavirus secuhominis
MELMDLLGL

SEQ ID NO: 53 - protein, 11 aa, Betapolyomavirus secuhominis
FFSVGGEALEL

SEQ ID NO: 54 - protein, 9 aa, Betapolyomavirus secuhominis
YCFDCFRQW

SEQ ID NO: 55 - protein, 9 aa, Betapolyomavirus secuhominis
TPHRHRVSA

SEQ ID NO: 56 - protein, 9 aa, Betapolyomavirus secuhominis
LLMGMYLDF

SEQ ID NO: 57 - protein, 11 aa, Betapolyomavirus secuhominis
VFLLMGMYLDF

SEQ ID NO: 58 - protein, 9 aa, Betapolyomavirus secuhominis
  MOD_RES (3)..(3): E or G
VEXSIQGGL

SEQ ID NO: 59 - protein, 10 aa, Betapolyomavirus secuhominis
  MOD_RES (4)..(4): I or L
TEVXGVTLMN

SEQ ID NO: 60 - protein, 9 aa, Betapolyomavirus secuhominis
ARIPLPNLN

SEQ ID NO: 61 - protein, 11 aa, Betapolyomavirus secuhominis
VKNPYPISFLL

SEQ ID NO: 62 - protein, 15 aa, Betapolyomavirus secuhominis
LPGDPDMMRYVDKYG

SEQ ID NO: 63 - protein, 15 aa, Betapolyomavirus secuhominis
LEVKTGVDSITEVEC

SEQ ID NO: 64 - protein, 15 aa, Betapolyomavirus secuhominis
DVCGMFTNRSGSQQW

SEQ ID NO: 65 - protein, 15 aa, Betapolyomavirus secuhominis
DAQVEEVRVFEGTEE

SEQ ID NO: 66 - protein, 10 aa, Betapolyomavirus secuhominis
QAVDTVAAKQ

SEQ ID NO: 67 - protein, 10 aa, Betapolyomavirus secuhominis
  MOD_RES (3)..(3): V or M
  MOD_RES (4)..(4): E, Q or G
MLXXRFNFLL

SEQ ID NO: 68 - protein, 9 aa, Betapolyomavirus secuhominis
LLLIWFRPV

SEQ ID NO: 69 - protein, 9 aa, Betapolyomavirus secuhominis
  MOD_RES (2)..(2): I or V
SXTEVECFL

SEQ ID NO: 70 - protein, 9 aa, Betapolyomavirus secuhominis
RLDLEISMY

SEQ ID NO: 71 - protein, 10 aa, Betapolyomavirus secuhominis
  MOD_RES (3)..(3): K or R
SVXVNLERKH

SEQ ID NO: 72 - protein, 9 aa, Betapolyomavirus secuhominis
  MOD_RES (5)..(5): K or R
AYLKXCKEL

SEQ ID NO: 73 - protein, 10 aa, Betapolyomavirus secuhominis
  SITE (1)..(1): May or may not be present
NLLMWEAVTV

SEQ ID NO: 74 - protein, 15 aa, Betapolyomavirus secuhominis
GSQQWRGLSRYFKVQ

SEQ ID NO: 75 - protein, 15 aa, Betapolyomavirus secuhominis
RGLSRYFKVQLRKRR

SEQ ID NO: 76 - protein, 15 aa, Betapolyomavirus secuhominis
RKAYLKKCKELHPDK

SEQ ID NO: 77 - protein, 15 aa, Betapolyomavirus secuhominis
WDEDLFCHEEMFASD

SEQ ID NO: 78 - protein, 15 aa, Betapolyomavirus secuhominis
CFRQWFGCDLTQEAL

SEQ ID NO: 79 - protein, 15 aa, Betapolyomavirus secuhominis
GGDEDKMKRMNFLYK

SEQ ID NO: 80 - protein, 15 aa, Betapolyomavirus secuhominis
KMKRMNFLYKKMEQG

SEQ ID NO: 81 - protein, 20 aa, Betapolyomavirus secuhominis
LNIPKKRYWLFKGPIDSGKT

SEQ ID NO: 82 - protein, 15 aa, Betapolyomavirus secuhominis
KRYWLFKGPIDSGKT

SEQ ID NO: 83 - protein, 15 aa, Betapolyomavirus secuhominis
VGPLCKGDNLYLSAV

SEQ ID NO: 84 - protein, 15 aa, Betapolyomavirus secuhominis
AYLDKNKAYPVECWV

SEQ ID NO: 85 - protein, 15 aa, Betapolyomavirus secuhominis
DMMRYVDRYGQLQTK

SEQ ID NO: 86 - protein, 9 aa, Betapolyomavirus secuhominis
SQHSTPPKK

SEQ ID NO: 87 - protein, 10 aa, Betapolyomavirus secuhominis
FPPNSDTLYC

SEQ ID NO: 88 - protein, 9 aa, Betapolyomavirus secuhominis
LLIKGGVEV

SEQ ID NO: 89 - protein, 10 aa, Artificial Sequence
  Description of Artificial Sequence Synthetic peptide
  MOD_RES (1)..(1): D or K
  MOD_RES (3)..(3): Q or K
  MOD_RES (9)..(9): K or R
  SITE (10)..(10): May or may not be present
XSXHSTPPXK

SEQ ID NO: 90 - protein, 9 aa, Artificial Sequence
  Description of Artificial Sequence Synthetic peptide
  MOD_RES (6)..(6): L or A
  MOD_RES (9)..(9): K or Q
AVDTVXAKX

SEQ ID NO: 91 - protein, 9 aa, Artificial Sequence
  Description of Artificial Sequence Synthetic peptide
  MOD_RES (4)..(4): I or F
  MOD_RES (8)..(8): T or R
CYCXDCFXQ

SEQ ID NO: 92 - protein, 9 aa, Artificial Sequence
  Description of Artificial Sequence Synthetic peptide
  MOD_RES (1)..(1): L or I
  MOD_RES (3)..(3): L or V
XPXMRKAYL

SEQ ID NO: 93 - protein, 9 aa, Artificial Sequence
  Description of Artificial Sequence Synthetic peptide
  MOD_RES (3)..(3): L or P
  MOD_RES (4)..(4): P or N
  MOD_RES (5)..(5): P or S
FPXXXDTLY

SEQ ID NO: 94 - protein, 9 aa, Artificial Sequence
  Description of Artificial Sequence Synthetic peptide
  MOD_RES (1)..(1): F or T
  MOD_RES (9)..(9): I or N
XLYCKEWPX

SEQ ID NO: 95 - protein, 9 aa, Artificial Sequence
  Description of Artificial Sequence Synthetic peptide
  MOD_RES (1)..(1): E or S
  MOD_RES (4)..(4): V or G
  MOD_RES (5)..(5): W or R
  MOD_RES (6)..(6): I or K
XPLXXXDCY

SEQ ID NO: 96 - protein, 10 aa, Artificial Sequence
  Description of Artificial Sequence Synthetic peptide
  MOD_RES (6)..(6): M or L
  MOD_RES (7)..(7): L or M
  MOD_RES (9)..(9): M or Q
VHCPCXXCXL

SEQ ID NO: 97 - protein, 9 aa, Artificial Sequence
  Description of Artificial Sequence Synthetic peptide
  MOD_RES (3)..(3): I or F
  MOD_RES (7)..(7): T or R
  MOD_RES (8)..(8): Q or E
YCXDCFXXW

SEQ ID NO: 98 - protein, 9 aa, Artificial Sequence
  Description of Artificial Sequence Synthetic peptide
  MOD_RES (3)..(3): L or M
  MOD_RES (8)..(8): E or D
LLXGMYLXF

SEQ ID NO: 99 - protein, 11 aa, Artificial Sequence
  Description of Artificial Sequence Synthetic peptide
  MOD_RES (2)..(2): F or L
  MOD_RES (10)..(10): E or D
VXLLMGMYLXF

SEQ ID NO: 100 - protein, 9 aa, Artificial Sequence
  Description of Artificial Sequence Synthetic peptide
  MOD_RES (1)..(1): I or V
  MOD_RES (3)..(3): E or G
  MOD_RES (6)..(6): Q or H
XEXSIXGGL

SEQ ID NO: 101 - protein, 10 aa, Artificial Sequence
  Description of Artificial Sequence Synthetic peptide
  MOD_RES (4)..(4): I, M, or L
  MOD_RES (6)..(6): I or V
  MOD_RES (8)..(8): S or L
  MOD_RES (10)..(10): L or N
TEVXGXTXMX

SEQ ID NO: 102 - protein, 15 aa, Artificial Sequence
  Description of Artificial Sequence Synthetic peptide
  MOD_RES (8)..(8): I or M
  MOD_RES (11)..(11): I or V
  MOD_RES (13)..(13): R or K
  MOD_RES (14)..(14): Q or Y
LPGDPDMXRYXDXXG

SEQ ID NO: 103 - protein, 15 aa, Artificial Sequence
  Description of Artificial Sequence Synthetic peptide
  MOD_RES (2)..(2): I or V
  MOD_RES (5)..(5): L or M
  MOD_RES (7)..(7): T or I
  MOD_RES (9)..(9): S or R
  MOD_RES (12)..(12): T or S
DXCGXFXNXSGXQQW

SEQ ID NO: 104 - protein, 10 aa, Artificial Sequence
  Description of Artificial Sequence Synthetic peptide
  MOD_RES (7)..(7): L or A
  MOD_RES (10)..(10): K or Q
QAVDTVXAKX

SEQ ID NO: 105 - protein, 10 aa, Artificial Sequence
  Description of Artificial Sequence Synthetic peptide
  MOD_RES (3)..(3): T, V, or M
  MOD_RES (4)..(4): E, D, or Q
  MOD_RES (8)..(8): H or F
  MOD_RES (9)..(9): I or L
MLXXRFNXXL

SEQ ID NO: 106 - protein, 9 aa, Artificial Sequence
  Description of Artificial Sequence Synthetic peptide
  MOD_RES (1)..(1): A or S
  MOD_RES (2)..(2): I or V
  MOD_RES (4)..(4): E or Q
XXTXVECFL

SEQ ID NO: 107 - protein, 9 aa, Artificial Sequence
  Description of Artificial Sequence Synthetic peptide
  MOD_RES (1)..(1): R or K
  MOD_RES (4)..(4): S or L
XLDXEISMY

SEQ ID NO: 108 - protein, 10 aa, Artificial Sequence
  Description of Artificial Sequence Synthetic peptide
  MOD_RES (3)..(3): K or R
  MOD_RES (8)..(8): E or R
SVXVNLEXKH

SEQ ID NO: 109 - protein, 9 aa, Artificial Sequence
  Description of Artificial Sequence Synthetic peptide
  MOD_RES (4)..(4): R or K
  MOD_RES (9)..(9): F or L
AYLXKCKEX

SEQ ID NO: 110 - protein, 9 aa, Artificial Sequence
  Description of Artificial Sequence Synthetic peptide
  SITE (1)..(1): May or may not be present
  MOD_RES (2)..(2): I or V
  MOD_RES (9)..(9): L or V
NXMWEAVTX

SEQ ID NO: 111 - protein, 15 aa, Artificial Sequence
  Description of Artificial Sequence Synthetic peptide
  MOD_RES (2)..(2): T or S
  MOD_RES (9)..(9): A or S
  MOD_RES (14)..(14): I or V
  MOD_RES (15)..(15): R or Q
GXQQWRGLXRYFKXX

SEQ ID NO: 112 - protein, 15 aa, Artificial Sequence
  Description of Artificial Sequence Synthetic peptide
  MOD_RES (4)..(4): A or S
  MOD_RES (9)..(9): I or V
  MOD_RES (10)..(10): R or Q
  MOD_RES (15)..(15): S or R
RGLXRYFKXXLRKRX

SEQ ID NO: 113 - protein, 15 aa, Artificial Sequence
  Description of Artificial Sequence Synthetic peptide
  MOD_RES (6)..(6): R or K
  MOD_RES (7)..(7): R or K
  MOD_RES (11)..(11): F or L
RKAYLXXCKEXHPDK

SEQ ID NO: 114 - protein, 15 aa, Artificial Sequence
  Description of Artificial Sequence Synthetic peptide
  MOD_RES (10)..(10): D or E
WDEDLFCHEXMFASD

SEQ ID NO: 115 - protein, 15 aa, Artificial Sequence
  Description of Artificial Sequence Synthetic peptide
  MOD_RES (3)..(3): T or R
  MOD_RES (8)..(8): L or C
  MOD_RES (12)..(12): E or Q
  MOD_RES (14)..(14): T or A
CFXQWFGXDLTXEXL

SEQ ID NO: 116 - protein, 15 aa, Artificial Sequence
  Description of Artificial Sequence Synthetic peptide
  MOD_RES (12)..(12): T or F
GGDEDKMKRMNXLYK

SEQ ID NO: 117 - protein, 15 aa, Artificial Sequence
  Description of Artificial Sequence Synthetic peptide
  MOD_RES (7)..(7): T or F
  MOD_RES (15)..(15): D or G
KMKRMNXLYKKMEQX

SEQ ID NO: 118 - protein, 20 aa, Artificial Sequence
  Description of Artificial Sequence Synthetic peptide
  MOD_RES (1)..(1): F or L
  MOD_RES (3)..(3): V or I
  MOD_RES (6)..(6): R or K
XNXPKXRYWLFKGPIDSGKT

SEQ ID NO: 119 - protein, 15 aa, Artificial Sequence
  Description of Artificial Sequence Synthetic peptide
  MOD_RES (1)..(1): R or K
XRYWLFKGPIDSGKT

SEQ ID NO: 120 - protein, 15 aa, Artificial Sequence
  Description of Artificial Sequence Synthetic peptide
  MOD_RES (7)..(7): A or G
  MOD_RES (9)..(9): S or N
VGPLCKXDXLYLSAV

SEQ ID NO: 121 - protein, 15 aa, Artificial Sequence
  Description of Artificial Sequence Synthetic peptide
  MOD_RES (7)..(7): N or K
  MOD_RES (15)..(15): I or V
AYLDKNXAYPVECWX

SEQ ID NO: 122 - protein, 15 aa, Artificial Sequence
  Description of Artificial Sequence Synthetic peptide
  MOD_RES (3)..(3): I or M
  MOD_RES (6)..(6): I or V
  MOD_RES (9)..(9): Q or G
DMXRYXDRXGQLQTK